(19) United States
(12) Patent Application Publication
(10) Pub. No.: US 2026/0272647 A1
(43) Pub. Date: Sep. 17, 2026

(54) DELIVERY APPARATUS FOR BALLOON EXPANDABLE PROSTHETIC HEART VALVE

(71) Applicant: Edwards Lifesciences Corporation, Irvine, CA (US)

(72) Inventors: Izaak Rosen, Irvine, CA (US); Sean Chow, Irvine, CA (US); Hannah Marie Glas, Costa Mesa, CA (US); Tri D. Tran, Fountain Valley, CA (US); Jeong Soo Lee, Yorba Linda, CA (US); Erik Bulman, Lake Forest, CA (US)

(73) Assignee: Edwards Lifesciences Corporation, Irvine, CA (US)

(21) Appl. No.: 19/659,164

(22) Filed: Apr. 27, 2026

Related U.S. Application Data
(63) Continuation of application No. PCT/US2024/054656, filed on Nov. 6, 2024.
(60) Provisional application No. 63/597,278, filed on Nov. 8, 2023.

(51) Int. Cl. A61F 2/24

(52) U.S. Cl. CPC A61F 2/2433

(57) ABSTRACT

Delivery apparatuses having valve positioning structures for a prosthetic heart valve are disclosed. As one example, a delivery apparatus can include a handle, a balloon catheter comprising a shaft coupled to the handle and a balloon connected to a distal end portion of the shaft, wherein the balloon is configured to be inflatable from a deflated state to an inflated state. A valve positioning structure is coupled to an exterior surface of the balloon and is configured to expand from a radially collapsed state to a radially expanded state when the balloon is inflated and then collapse back to the radially collapsed state when the balloon is deflated.

### CROSS-REFERENCE TO RELATED APPLICATION

This application is a continuation of PCT Application No. PCT/US2024/054656, filed November 6, 2024, which claims the benefit of U.S. Provisional Patent Application No. 63/597,278, filed on November 8, 2023, wherein each of the above-referenced applications is incorporated by reference herein in its entirety.

### FIELD

The present disclosure relates to delivery systems for prosthetic heart valves, and in particular to delivery systems with inflatable balloons for implanting prosthetic heart valves.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient’s vasculature (for example, through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

### SUMMARY

Described herein are prosthetic heart valves, delivery apparatus, and methods for implanting prosthetic heart valves. The disclosed prosthetic heart valves, delivery apparatus, and methods can, for example, provide improved positioning of a prosthetic heart valve on a delivery apparatus, for example, during advancement of the delivery apparatus through a patient’s vasculature, among other things. As such, the devices and methods disclosed herein can, among other things, provide improved delivery apparatuses for balloon-expandable prosthetic valves.

A delivery apparatus for a prosthetic implant can comprise a handle, one or more shafts coupled to the handle, and an inflatable balloon. The delivery apparatus can further include one or more of the following features.

In some examples, a delivery apparatus can comprise a valve positioning structure positioned along a distal end portion of the balloon.

In some examples, a valve positioning structure comprises an integral nose cone that is connected to the distal end portion of a shaft distal to the balloon.

In some examples, a valve positioning structure is connected to a nose cone.

In some examples, an inner surface of the valve positioning structure is bonded to an outer surface of the nose cone.

In some examples, a valve positioning structure is positioned along a proximal end portion of the balloon.

In some examples, a delivery apparatus comprises a first shaft and a second shaft extending coaxially over the first shaft, wherein a first end portion of a valve positioning structure is connected to a distal end portion of the second shaft.

In some examples, the second shaft is movable axially relative to the first shaft and the balloon, and the valve positioning structure is configured to move the prosthetic valve relative to the balloon from an offset position to a valve mounting portion of the balloon.

In some examples, a maximum outer diameter of a valve positioning structure is greater than an outer diameter of a prosthetic valve mounted on the delivery apparatus in the radially compressed state.

In some examples, a valve positioning structure is elastically expandable and collapsible upon inflation and deflation of the balloon, respectively.

In some examples, a valve positioning structure stretches radially and circumferentially when the balloon is inflated and contracts radially and circumferentially when the balloon is deflated.

In some examples, a valve positioning structure is made of an elastomer.

In some examples, a valve positioning structure comprises an inner member and an outer member positioned radially outwards of the inner member.

In some examples, the inner member comprises a base and a plurality of prongs extending axially from the base, wherein the prongs extend at least partially over the exterior of the balloon and are configured to transition from the radially collapsed state to the radially expanded state when the balloon is inflated, and wherein the outer member is configured to urge the prongs back to the radially collapsed state when the balloon is deflated.

In some examples, the inner member comprises a material that is more rigid than the outer member.

In some examples, the outer member comprises an elastomeric band or sleeve.

In some examples, the outer member comprises a coiled member.

In some examples, the outer member is a closed annular band.

In some examples, the outer member is made of a shape memory material and is shaped set in a radially collapsed state.

In some examples, a valve positioning structure comprises a plurality of axially extending rods and an annular, elastomeric layer extending around the rods.

In some examples, the rods are made of a first material and the elastomeric layer is made of a second material, wherein the first material is stiffer than the second material.

In some examples, the elastomeric layer and the rods comprise different materials that are coextruded together.

In some examples, a delivery apparatus comprises a first valve positioning structure extending over a distal end portion of the balloon and a second valve positioning structure extending over a proximal end portion of the balloon.

In some examples, a delivery apparatus comprises one or more of the components recited in Examples 1-139 below.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 is a side view of a prosthetic heart valve, according to one example.

FIG. 2A is a side view of a delivery apparatus for a prosthetic heart valve, according to one example.

FIG. 2B is side view of a distal end portion of a delivery apparatus for a prosthetic heart valve, according to one example.

FIG. 3A is a cross-sectional side view of a distal end portion of the delivery apparatus of FIG. 2A having a distal valve positioning structure, according to one example, shown with a balloon in an unexpanded configuration and a prosthetic valve in a radially crimped state on the balloon.

FIG. 3B is a cross-sectional side view of the distal end portion of the delivery apparatus of FIG. 3A, shown with the balloon in an expanded configuration and the prosthetic valve in a radially expanded state.

FIG. 4 is a side view of a distal end portion of the delivery apparatus of FIG. 2A having a distal valve positioning structure, according to one example, shown with a balloon in an uninflated configuration.

FIG. 5 is a perspective view of the valve positioning structure of the delivery apparatus of FIG. 4.

FIG. 6 is a perspective view of a valve positioning structure of a delivery apparatus, according to one example.

FIG. 7A is a perspective view of a valve positioning structure of a delivery apparatus including a nose cone, according to one example.

FIG. 7B is a cross-sectional view of the valve positioning structure of FIG. 7A.

FIG. 8 is a side view of a distal end portion of the delivery apparatus of FIG. 2A having proximal and distal valve positioning structures, according to one example, shown with a balloon in an unexpanded configuration.

FIG. 9 is a side view of the distal end portion of the delivery apparatus of FIG. 8, shown with the balloon in an unexpanded configuration and a prosthetic valve in a radially crimped state on the balloon.

FIG. 10 is a perspective view of a valve positioning structure of a delivery apparatus having a first member and a second member, according to one example.

FIG. 11 is a perspective view of the first member of the valve positioning structure of FIG. 10.

FIG. 12 is a perspective view of the second member of the valve positioning structure of FIG. 10, according to one example.

FIG. 13 is a perspective view of a first member of a valve positioning structure of a delivery apparatus, according to one example.

FIG. 14 is a perspective view of a valve positioning structure of a delivery apparatus, according to one example.

FIG. 15 is a side view of a distal end portion of the delivery apparatus of FIG. 2A having proximal and distal valve positioning structures, according to one example, shown with a balloon in an unexpanded configuration.

FIG. 16A is a side view of a distal end portion of the delivery apparatus of FIG. 2A having proximal and distal valve positioning structures, according to one example, shown with a balloon in an unexpanded configuration and a prosthetic valve in a radially crimped state on the balloon.

FIG. 16B is a side view of the distal end portion of the delivery apparatus of FIG. 16A, shown with the balloon in an expanded configuration and the prosthetic valve in a radially expanded state.

FIG. 17A is an end view of a biasing member for a valve positioning structure shown in a radially collapsed state, according to one example.

FIG. 17B is an end view of the biasing member of FIG. 17B, shown in a radially expanded state.

FIG. 18A is an end view of a valve positioning structure of a delivery apparatus shown in a radially collapsed state, according to one example.

FIG. 18B is an end view of the valve positioning structure of FIG. 18A, shown in a radially expanded state.

FIG. 19A is a perspective view of a valve positioning structure, according to one example.

FIG. 19B is a perspective view of the biasing member of the valve positioning structure of FIG. 19A.

FIG. 20 is a perspective view of a valve positioning structure mounted on a shaft of a delivery apparatus, according to one example.

FIG. 21 is a cross-sectional view of the valve positioning structure of FIG. 21.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (for example, out of the patient’s body), while distal motion of the device is motion of the device away from the user and toward the implantation site (for example, into the patient’s body). The terms “longitudinal” and “axial” refer to an axis extending in the proximal and distal directions, unless otherwise expressly defined.

As used herein, “for example” means “for example,” and “i.e.” means “that is.”

### Overview of the Disclosed Technology

Described herein are examples of a delivery apparatus that can be used to navigate a subject’s vasculature to deliver an implantable, expandable medical device (for example, a prosthetic heart valve), tools, agents, or other therapy to a location within the body of a subject. Examples of procedures in which the steerable catheters are useful include neurological, urological, gynecological, fertility (for example, in vitro fertilization, artificial insemination), laparoscopic, arthroscopic, transesophageal, transvaginal, transvesical, transrectal, and procedures including access in any body duct or cavity. Particular examples include placing implants, including stents, grafts, embolic coils, and the like; positioning imaging devices and/or components thereof, including ultrasound transducers; and positioning energy sources, for example, for performing lithotripsy, RF sources, ultrasound emitters, electromagnetic sources, laser sources, thermal sources, and the like.

Prosthetic valves disclosed herein can be radially compressible and expandable between a radially compressed state and a radially expanded state. Thus, the prosthetic valves can be crimped on or retained by an implant delivery apparatus in the radially compressed state while being advanced through a patient’s vasculature on the delivery apparatus. The prosthetic valve can be expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

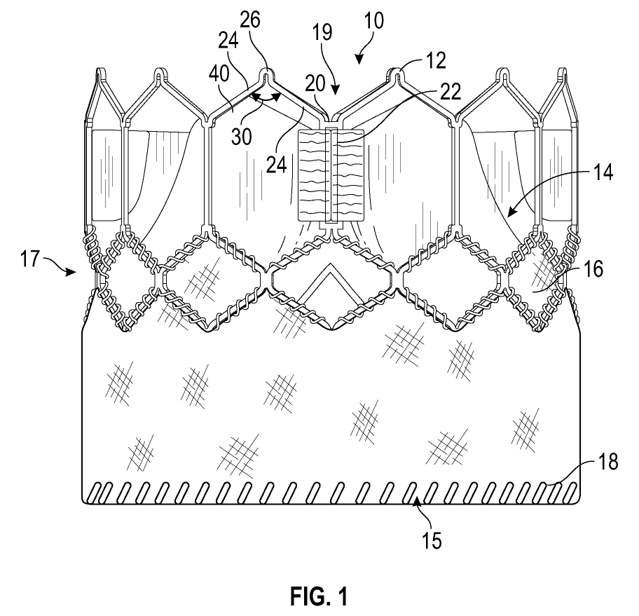

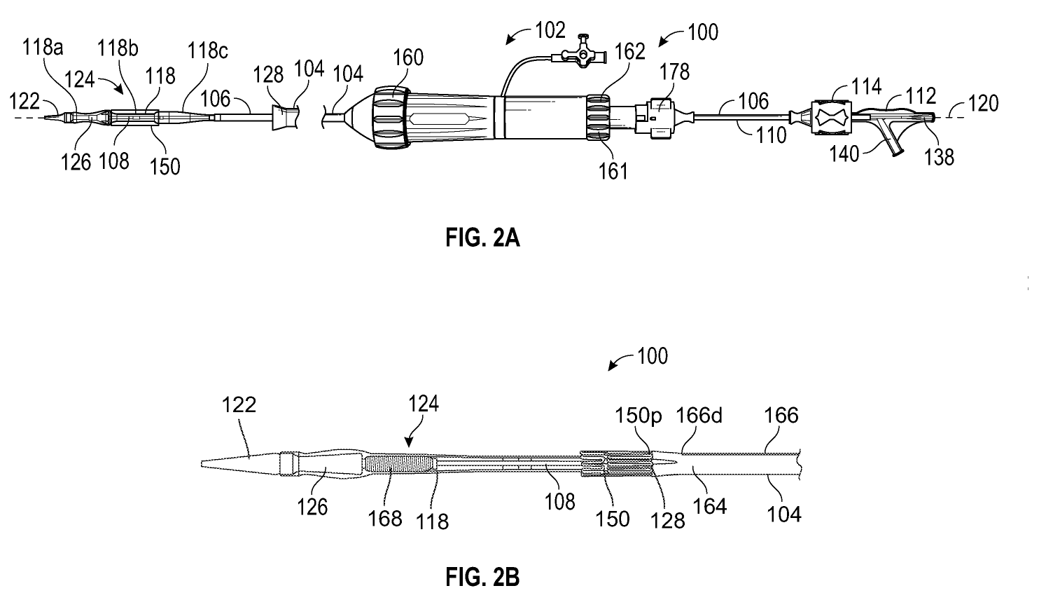

As introduced above, some prosthetic heart valves, such as the example prosthetic heart valve depicted in FIG. 1, can be retained by a delivery apparatus in the radially compressed state and advanced through a patient’s vasculature, such as to a native heart valve, by the delivery apparatus, such as the example delivery apparatus shown in FIG. 2A. The prosthetic valve can be expanded to the radially expanded state once the prosthetic valve reaches the implantation site, for example, by expanding a balloon of the delivery apparatus.

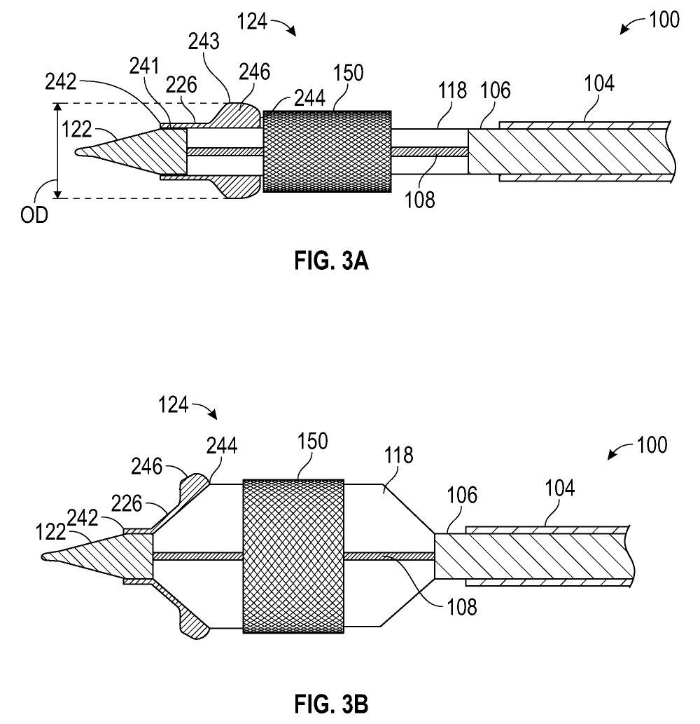

In some examples, the delivery apparatus can include structures that help position the prosthetic heart valve on the distal end of the delivery apparatus. For example, as shown in FIGS. 3-20B, a distal valve positioning structure (for example, a flex tip) and/or a proximal valve positioning structure (for example, a shoulder) can be coupled to an external surface of the balloon of the delivery apparatus to hold or retain the prosthetic heart valve in an axially fixed position relative to the delivery apparatus. In some examples, the structures can be configured with tapered surfaces and/or cross-sectional sizes that improve the ability of the delivery apparatus to advance the prosthetic heart valve through the patient’s vasculature. As a result, the devices and methods disclosed herein can, among other things, improve delivery apparatuses for prosthetic heart valves by improving the positioning of the prosthetic heart valve relative to the delivery apparatus and improving the ability for the delivery apparatus to advance through a patient’s vasculature.

### Examples of the Disclosed Technology

FIG. 1 shows a prosthetic heart valve 10 (prosthetic valve), according to one example. Any of the prosthetic valves disclosed herein are adapted to be implanted in the native aortic annulus, although in other examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In another example, the disclosed prosthetic valves can be implanted within a docking device implanted within or at the native mitral valve, such as disclosed in PCT Publication No. WO2020/247907, which is incorporated by reference herein. In another example, the disclosed prosthetic valves can be implanted within a docking device implanted within the superior or inferior vena cava for replacing the function of a diseased tricuspid valve, such as disclosed in U.S. Publication No. 2019/0000615, which is incorporated by reference herein.

The prosthetic valve 10 can include a stent or frame 12, a valvular structure 14, an inner skirt 16, and a perivalvular outer sealing member or outer skirt 18. The prosthetic valve 10 can have an inflow end portion 15, an intermediate portion 17, and an outflow end portion 19. The inner skirt 16 can be arranged on and/or coupled to an inner surface of the frame 12 while the outer skirt 18 can be arranged on and/or coupled to an outer surface of the frame 12.

The valvular structure 14 can comprise three leaflets 40, collectively forming a leaflet structure, which can be arranged to collapse in a tricuspid arrangement, although in other examples there can be greater or fewer number of leaflets (for example, one or more leaflets 40). The leaflets 40 can be secured to one another at their adjacent sides to form commissures 22 of the leaflet structure 14. The lower edge of valvular structure 14 can have an undulating, curved scalloped shape and can be secured to the inner skirt 16 by sutures (not shown). In some examples, the leaflets 40 can be formed of pericardial tissue (for example, bovine pericardial tissue), biocompatible synthetic materials, or various other suitable natural or synthetic materials as known in the art and described in U.S. Patent No. 6,730,118, which is incorporated by reference herein.

The frame 12 can be radially compressible (collapsible) and expandable (for example, expanded configuration shown in FIG. 1) and comprise a plurality of interconnected struts 24. A plurality of apices 26 that are spaced circumferentially apart are formed at the inflow end portion 15 and the outflow end portion 19 of the frame 12 (only the apices 26 at the outflow end portion 19 are visible in FIG. 1). Each apex 26 is formed at a junction between two angled struts 24 at either the inflow end portion 15 or the outflow end portion 19. FIG. 1 depicts a known frame design with apices 26 that form a U-shaped bend between the two angled struts 24. In some examples, an angle 30 between the two angled struts 24, connected at the apex 26, can be in a range of 90 to 120 degrees.

The frame 12 can be formed with a plurality of circumferentially spaced slots, or commissure windows 20 that are adapted to mount the commissures 22 of the valvular structure 14 to the frame. The frame 12 can be made of any of various suitable plastically-expandable materials (for example, stainless steel, etc.) or self-expanding materials (for example, Nitinol). When constructed of a plastically-expandable material, the frame 12 (and thus the prosthetic valve 10) can be crimped to a radially collapsed configuration on a delivery catheter or apparatus and then expanded inside a patient by an inflatable balloon or equivalent expansion mechanism. When constructed of a self-expandable material, the frame 12 (and thus the prosthetic valve 10) can be crimped to a radially collapsed configuration and restrained in the collapsed configuration by insertion into a sheath or equivalent mechanism of a delivery catheter. Once inside the body, the prosthetic valve can be advanced from the sheath, which allows the prosthetic valve to expand to its functional size.

Suitable plastically-expandable materials that can be used to form the frame 12 include, without limitation, stainless steel, a biocompatible, high-strength alloys (for example, a cobalt-chromium or a nickel-cobalt-chromium alloys), polymers, or combinations thereof. In particular examples, frame 12 is made of a nickel-cobalt-chromium-molybdenum alloy, such as MP35N® alloy (SPS Technologies, Jenkintown, Pennsylvania), which is equivalent to UNS R30035 alloy (covered by ASTM F562-02). MP35N® alloy/UNS R30035 alloy comprises 35% nickel, 35% cobalt, 20% chromium, and 10% molybdenum, by weight. Additional details regarding the prosthetic valve 10 and its various components are described in WIPO Patent Application Publication No. WO 2018/222799, which is incorporated herein by reference.

FIG. 2A shows a delivery apparatus 100, according to an example, that can be used to implant an expandable prosthetic heart valve (for example, prosthetic heart valve 10 of FIG. 1 or any of the other prosthetic heart valves described herein). In some examples, the delivery apparatus 100 is specifically adapted for use in introducing a prosthetic valve into a heart.

The delivery apparatus 100 in the illustrated example of FIG. 2A is a balloon catheter comprising a handle 102 and a steerable, outer shaft 104 extending distally from the handle 102. The delivery apparatus 100 can further comprise an intermediate shaft 106 (which also may be referred to as a balloon shaft) that extends proximally from the handle 102 and distally from the handle 102, the portion extending distally from the handle 102 also extending coaxially through the outer shaft 104. Additionally, the delivery apparatus 100 can further comprise an inner shaft 108 extending distally from the handle 102 coaxially through the intermediate shaft 106 and the outer shaft 104 and proximally from the handle 102 coaxially through the intermediate shaft 106.

The outer shaft 104 and the intermediate shaft 106 can be configured to translate (for example, move) longitudinally, along a central longitudinal axis 120 of the delivery apparatus 100, relative to one another to facilitate delivery and positioning of a prosthetic valve at an implantation site in a patient’s body.

The intermediate shaft 106 can include a proximal end portion 110 that extends proximally from a proximal end of the handle 102, to an adaptor 112. A rotatable knob 114 can be mounted on the proximal end portion 110 and can be configured to rotate the intermediate shaft 106 around the central longitudinal axis 120 and relative to the outer shaft 104.

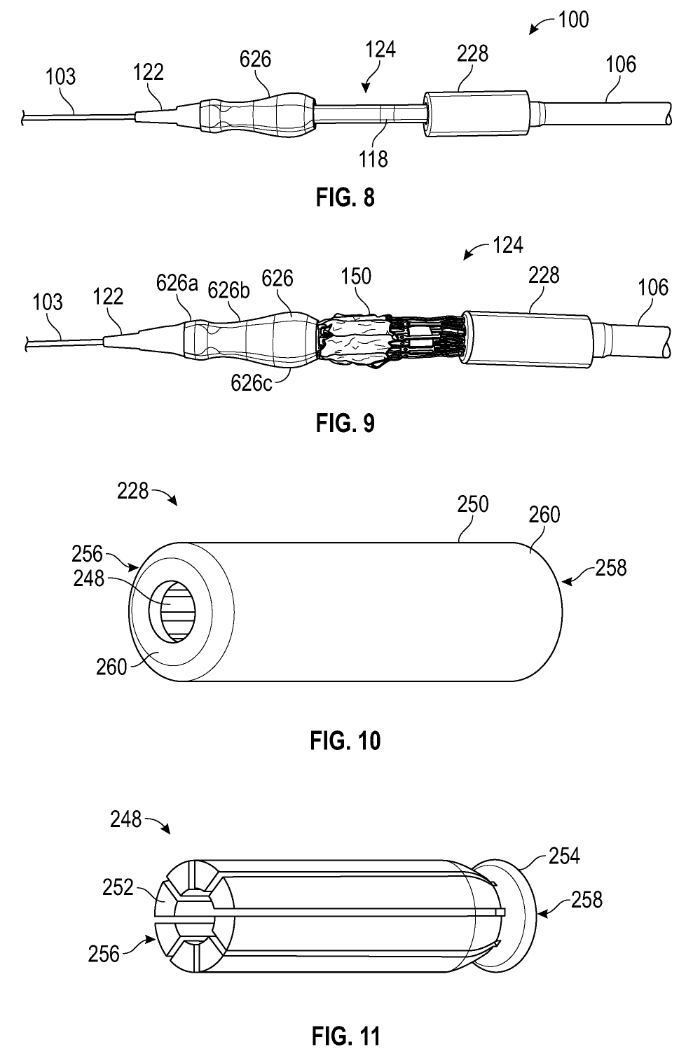

The adaptor 112 can include a first port 138 configured to receive a guidewire 103 (see for example, FIGS. 8-9) therethrough and a second port 140 configured to receive fluid (for example, inflation fluid) from a fluid source. The second port 140 can be fluidly coupled to an inner lumen of the intermediate shaft 106.

The intermediate shaft 106 can further include a distal end portion that extends distally beyond a distal end of the outer shaft 104 when a distal end of the outer shaft 104 is positioned away from an inflatable balloon 118 of the delivery apparatus 100. A distal end portion of the inner shaft 108 can extend distally beyond the distal end portion of the intermediate shaft 106.

The balloon 118 can extend over the inner shaft 108. The balloon 118 can include a distal end portion 118a, an intermediate portion 118b, and a proximal portion 118c.

In some examples, the distal end portion 118a of the balloon 118 can be coupled to a distal end of the delivery apparatus 100, such as to a nose cone 122 (as shown in FIG. 2A), or to an alternate component at the distal end of the delivery apparatus 100 (for example, a distal shoulder). The proximal end portion 118c of the balloon 118 can be coupled to a distal end portion of the intermediate shaft 106. The intermediate portion 118b of the balloon 118 and the inner shaft can form or define a valve mounting portion 124 of a distal end portion of the delivery apparatus 100. The distal end portion 118a of the balloon 118 can overlay a distal shoulder 126 of the delivery apparatus 100. The valve mounting portion 124 and the intermediate portion of the balloon 118 can be configured to receive a prosthetic heart valve in a radially compressed state. For example, as shown schematically in FIG. 2A, a prosthetic heart valve 150 (which can be one of the prosthetic valves described herein, for example, prosthetic heart valve 10) can be mounted around the balloon 118, at the valve mounting portion 124 of the delivery apparatus 100. The intermediate portion 118b of the balloon alternatively can be referred to as a valve mounting portion of the balloon 118.

The balloon shoulder assembly, including the distal shoulder 126, is configured to maintain the prosthetic heart valve 150 (or other medical device) at a fixed position on the balloon 118 during delivery through the patient’s vasculature.

The outer shaft 104 can include a distal tip portion 128 mounted on its distal end. The outer shaft 104 and the intermediate shaft 106 can be translated axially relative to one another to position the distal tip portion 128 adjacent to a proximal side of the valve mounting portion 124, when the prosthetic valve 150 is mounted in the radially compressed state on the valve mounting portion 124 (as shown in FIG. 2A) and during delivery of the prosthetic valve to the target implantation site. As such, the distal tip portion 128 can be configured to resist movement of the prosthetic valve 150 relative to the balloon 118 proximally, in the axial direction, when the distal tip portion 128 is arranged adjacent to a proximal side of the valve mounting portion 124.

An annular space can be defined between an outer surface of the inner shaft 108 and an inner surface of the intermediate shaft 106 and can be configured to receive fluid from a fluid source via the second port 140 of the adaptor 112. The annular space can be fluidly coupled to a fluid passageway formed between the outer surface of the distal end portion of the inner shaft 108 and an inner surface of the balloon 118. As such, fluid from the fluid source can flow to the fluid passageway from the annular space to inflate the balloon 118 and radially expand and deploy the prosthetic valve 150.

An inner lumen of the inner shaft can be configured to receive a guidewire therethrough, for navigating the distal end portion of the delivery apparatus 100 to the target implantation site.

The handle 102 can include a steering mechanism configured to adjust the curvature of the distal end portion of the delivery apparatus 100. In the illustrated example, for example, the handle 102 includes an adjustment member, such as the illustrated rotatable knob 160, which in turn is operatively coupled to the proximal end portion of a pull wire. The pull wire can extend distally from the handle 102 through the outer shaft 104 and has a distal end portion affixed to the outer shaft 104 at or near the distal end of the outer shaft 104. Rotating the knob 160 can increase or decrease the tension in the pull wire, thereby adjusting the curvature of the distal end portion of the delivery apparatus 100. Further details on steering or flex mechanisms for the delivery apparatus can be found in U.S. Patent No. 9,339,384, which is incorporated by reference herein.

The handle 102 can further include an adjustment mechanism 161 including an adjustment member, such as the illustrated rotatable knob 162, and an associated locking mechanism including another adjustment member, configured as a rotatable knob 178. The adjustment mechanism 161 is configured to adjust the axial position of the intermediate shaft 106 relative to the outer shaft 104 (for example, for fine positioning at the implantation site). Further details on the delivery apparatus 100 can be found in WIPO Publication No. WO2022/046585, which are incorporated by reference herein.

FIG. 2A shows an example of the delivery apparatus 100 being used for “on-balloon” delivery. For such on-balloon delivery, the prosthetic valve 150 is radially compressed directly onto the valve mounting portion 124 of the balloon 118 prior to insertion of the prosthetic valve and the delivery apparatus into a patient’s vasculature.

FIG. 2B shows an example of the delivery apparatus 100 being used for “off-balloon” delivery. For such off-balloon delivery, the prosthetic valve 150 is radially crimped offset from the valve mounting portion 124 (referred to as an offset position), such as on the balloon catheter shaft 106 and/or on a proximal end portion of the balloon. The delivery apparatus 100 and the prosthetic valve 150 are inserted through an introducer sheath and into the patient’s vasculature with the prosthetic valve in this offset position. Once inside the patient’s vasculature (for example, within the descending aorta), the prosthetic valve 150 can be moved from the offset position to the valve mounting portion 124 of the balloon 118. This can be accomplished by moving the shaft 106 proximally relative to the shaft 104 or moving the shaft 104 distally relative to the shaft 106 while applying a distally-directed force against a proximal end 150p of the prosthetic valve with a distal end of the tip portion 128. In some examples, the adjustment mechanism 161 can be used to move the shaft 106 relative to the shaft 104 for repositioning the prosthetic valve. Thereafter, the delivery apparatus 100 can be further advanced to the target implantation site (for example, the native aortic annulus) and the prosthetic valve 150 can be deployed. The delivery apparatus 100 optionally can include a valve mounting member 168 positioned inside the balloon along the valve mounting portion 124 to help maintain the position of the prosthetic valve on the balloon.

FIG. 2B also shows a pull wire 166 (which can extend through a lumen of the shaft 104) having a distal end 166d that is fixed relative to a steerable section 164 of the shaft 104. The proximal end of the pull wire 166 can be operatively connected to the adjustment knob 160 (FIG. 2B) for adjusting the tension in the pull wire for controlling the curvature of the steerable section 164 of the shaft 104. Further details regarding a delivery apparatus used for off-balloon delivery of a prosthetic valve are disclosed in U.S. Publication No. U.S. Publication No. 2013/0030519, which is incorporated herein by reference.

When the prosthetic valve 150 is retained on the valve mounting portion 124, a distal end of the prosthetic valve 150 is positioned adjacent to a distal end of the valve mounting portion 124, that is, adjacent to the distal shoulder 126. As such, the distal shoulder 126 can be configured to resist movement of the prosthetic valve 150 relative to the balloon 118 distally, in the axial direction, for example, when the balloon 118 is initially expanded. In some examples, as shown, the distal shoulder 126 is positioned within the balloon 118, such that the balloon 118 expands radially outward and away from the distal shoulder 126 as the balloon 118 radially expands and deploys the prosthetic heart valve 150. The distal shoulder 126 also functions to protect and shield the leading edge (the distal edge) of the prosthetic valve 150 from contacting native anatomy as the delivery apparatus is advanced through the patient’s vasculature.

Additionally, during delivery of the prosthetic valve 150, the outer shaft 104 can be positioned relative to the intermediate shaft 106 such that the distal tip portion 128 is positioned adjacent to a proximal end of the valve mounting portion 124 and adjacent to a proximal end of the prosthetic valve 150. In some examples, the distal end of the tip portion 128 can abut the proximal end of the prosthetic valve. As such, in some examples, the distal tip portion 128 is also referred to herein as a “proximal shoulder.” In this position, the outer shaft 104 is disposed around and covers a portion of the balloon 118. The proximal shoulder 128 can be configured to resist movement of the prosthetic valve 150 relative to the balloon 118 proximally, in the axial direction, for example, as the delivery apparatus and prosthetic heart valve are advanced through the patient’s vasculature and/or an introducer sheath. However, prior to expanding the balloon 118, the outer shaft 104 must be retracted proximally relative to the intermediate shaft 106 to enable the balloon 118 to be fully inflated (for example, without interference with the outer shaft 104). This is due in part to the outer shaft 104 and/or the proximal shoulder 128 not being expandable when the balloon 118 is radially expanded. Also, as noted above, for off-balloon delivery, the distal tip portion 128 of the shaft 104 can be used to push the prosthetic valve 150 onto the valve mounting portion 124 once the prosthetic valve and the distal end portion of the delivery apparatus are inserted into the patient’s vasculature.

Structures positioned inside of a balloon, such as the distal shoulder 126, while effective, involve relatively complex manufacturing steps. For example, once the distal shoulder is positioned inside of the balloon, additional steps may be required to form or shape the balloon around the distal shoulder.

Moreover, when deploying the prosthetic valve, the prosthetic valve desirably is positioned to be substantially coaxial with the annulus of the native heart valve so that the prosthetic valve can be evenly expanded and securely anchored within the annulus. In some circumstances, despite the initial position of the heart valve being coaxial with the native annulus, such coaxiality may be disturbed or lost after retracting the outer shaft 104 off the balloon 118 just prior to balloon inflation. This can occur if the steerable section 164 of the shaft 104 is retained in a curved state via increased tension in the pull wire while the shaft 104 is retracted. Retracting the shaft 104 retracts the steerable section 164 farther away from the balloon 118 and the prosthetic valve 150. Without the structural support of the steerable section 164, the distal end portion of the balloon catheter shaft 106 and the distal end portion of the inner shaft 108 (where the balloon 118 and the prosthetic valve 150 are mounted) may deflect slightly relative to the steerable section 164. As a result, the prosthetic valve 150 may no longer be coaxial with the native annulus.

Thus, in some examples, it may be desirable for structures that help maintain the axial position of a prosthetic valve relative to a delivery apparatus to be coupled to an outer surface of a balloon of a delivery apparatus (for example, balloon 118) to facilitate insertion of a prosthetic heart valve through a patient’s vasculature and an introducer sheath, improve the positioning of the prosthetic heart valve relative to the balloon 118 during delivery, and do so without requiring translation of the outer shaft 104 prior to inflating the balloon 118, among other things. For example, these structures can be configured to expand as the balloon expands, and in some examples, these structures can help maintain the position of the prosthetic valve relative to the delivery apparatus during radial expansion of the prosthetic valve. In some examples, these structures can be used in lieu of the distal shoulder 126 and/or the proximal shoulder 128.

FIGS. 3A-3B illustrate the valve mounting portion 124 of the distal end portion of the delivery apparatus 100 that includes an expandable distal shoulder 226, in lieu of the distal shoulder 126. Unlike the distal shoulder 126 which is positioned within the balloon 118, the expandable distal shoulder 226 is positioned radially outside of the balloon 118, for example, on an outer surface of the balloon 118. In FIG. 3, the distal end portion of the outer shaft 104 is shown without the enlarged tip portion 128 (which can also function as a proximal shoulder); however, it should be understood that the outer shaft 104 in this example can include the tip portion 128. In lieu of or in addition to the tip portion 128, the delivery apparatus 100 of FIGS. 3A-3B can include any of the other examples of proximal shoulders disclosed herein.

The expandable distal shoulder 226 may be coupled to the nose cone 122, such that the expandable distal shoulder 226 is positioned radially outwards of the balloon 118. In some examples, a distal portion of the balloon 118 can be coupled to an outer surface of the nose cone 122, such that a portion of the balloon 118 is positioned radially between the outer surface of the nose cone 122 and an inner surface of the expandable distal shoulder 226. In some examples, the balloon 118 can be coupled to an inner surface of the nose cone 122.

The expandable distal shoulder 226 includes a distal portion 241 defining a first (or distal) end 242 of the shoulder and a proximal portion 243 defining a second (or proximal) end 244 of the shoulder. As shown in FIGS. 3A-3B, the distal portion 241 is coupled to the nose cone 122. The distal portion 241 of the shoulder 226 can be fixed to the outer surface of the nose cone 122, such as with an adhesive, over molding, welding, mechanical fasteners, or other attachment means. The proximal portion 243 extends over and around a portion of the balloon 118 such that the proximal end 244 is positioned adjacent to the distal end of the valve mounting portion 124, and such that the second end 244 is adjacent to or in contact with the prosthetic valve 150, when the prosthetic valve 150 is mounted on the delivery apparatus 100. As such, the second end 244 of the expandable distal shoulder 226 is configured to resist movement of the prosthetic valve 150 relative to the balloon 118 distally, in the axial direction, for example, during delivery through the patient’s vasculature or as the balloon 118 is expanded.

FIG. 3A illustrates the delivery apparatus 100 in a delivery state in which the balloon 118 is in a collapsed (uninflated) configuration and FIG. 3B illustrates the delivery apparatus 100 in a deployed state in which the balloon is inflated and the prosthetic valve 150 is radially expanded. As shown, in the deployed state, the balloon 118, the prosthetic valve 150, and the proximal portion 243 of the expandable distal shoulder 226 are in a radially expanded configuration. The expandable distal shoulder 226 comprises a material that allows the portion of the distal shoulder 226 on the inflatable portion of the balloon 118 to expand in the radial direction upon inflation of the balloon, such that an outer diameter of the expandable distal shoulder 226 expands as the balloon 118 is expanded.

The expandable distal shoulder 226 is configured to return to the radially collapsed delivery state when the balloon 118 is deflated, for example, prior to removal of the delivery apparatus 100 from the patient’s vasculature. The expandable distal shoulder 226 comprises a material that allows the expanded portion of the distal shoulder 226 to contract or collapse in the radial direction upon removal of the expansion force of the balloon, such that an outer diameter of the proximal portion 243 of the expandable distal shoulder 226 decreases as the balloon 118 is collapsed (deflated). In this way, the expandable distal shoulder 226 does not interfere with the patient’s vasculature upon removal of the delivery apparatus 100 from the patient.

In some examples, the expandable distal shoulder 226 can comprise an elastic and/or resilient material, such as natural rubber or any of various synthetic elastomers, such as polyurethane (for example, Tecoflex™ or NueSoft™), styrene ethylene butylene styrene (SEBS), styrene butadiene styrene (SBS), styrene isoprene styrene (SIS), or thermoplastic vulcanizate (TPV) (for example, Santoprene). When made of such materials, the shoulder 226 can be referred to as an elastic sleeve or an elastomeric sleeve. As such, when the balloon is inflated, the proximal portion 243 of the shoulder 226 can expand radially and circumferentially to its expanded shape shown in FIG. 3B. When the balloon is deflated, the proximal portion 243 can contract radially and circumferentially back to its unexpanded shape shown in FIG. 3A under its own resiliency.

In some examples, the distal portion 241 can be cylindrical and the proximal portion 243 can have variable diameter that defines the maximum outer diameter of the shoulder. For example, as shown, the outer diameter of the expandable distal shoulder 226 generally increases in the proximal direction, such that an outer diameter OD of the expandable distal shoulder 226 at an intermediate location (for example, a location between the first end 242 and the second end 244) is greater than an outer diameter at the first end 242. In some examples, the maximum outer diameter of the expandable distal shoulder 226 is located at the second end 244. In some examples, as depicted in FIG. 3A, the maximum outer diameter of the expandable distal shoulder 226 is slightly spaced away from the second end 244 in the distal direction. In some examples, the maximum outer diameter of the expandable distal shoulder 226 can be positioned at different axial locations depending on whether the expandable distal shoulder 226 is in a relaxed configuration (FIG. 3A) or an expanded configuration (FIG. 3B).

In some examples, the tapered outer profile of the expandable distal shoulder 226 enables the delivery apparatus 100 to be more easily advanced through a patient’s vasculature in a manner that protects against undesirable contact between the leading edge (the distal end) of the prosthetic heart valve 150 and the patient’s vasculature. For example, as shown in FIG. 3A, the outer diameter of the expandable distal shoulder 226 at the distal end 244 can be slightly larger than an outer diameter of the prosthetic heart valve 150 in its radially compressed state on the balloon.

In some examples, the proximal portion 243 of the expandable distal shoulder 226 includes a protuberance 246 that extends around the circumference of the expandable distal shoulder 226. The protuberance 246 is a region of the expandable distal shoulder 226 having an increased outer diameter, for example, as compared to the outer diameter of the expandable distal shoulder 226 at the first end 242. The protuberance 246 in the illustrated example has a rounded edge at the proximal end 244 of the shoulder, a generally cylindrical intermediate portion and a tapered distal portion that decreases in diameter from the intermediate portion to the distal portion 241.

The protuberance 246 can have an outer diameter that is slightly larger than an outer diameter of the valve 150 crimped to the valve mounting portion 124 of the deliver apparatus 100. In this way, the protuberance 246 can be configured to help prevent the distal end of the valve 150 from interfering with native anatomy as the delivery apparatus 100 is advanced through a patient’s vasculature and/or contacting the inner surface of an introducer sheath. In some examples, the maximum outer diameter of the expandable distal shoulder 226 is located at the protuberance 246, as shown in FIGS. 3A-3B.

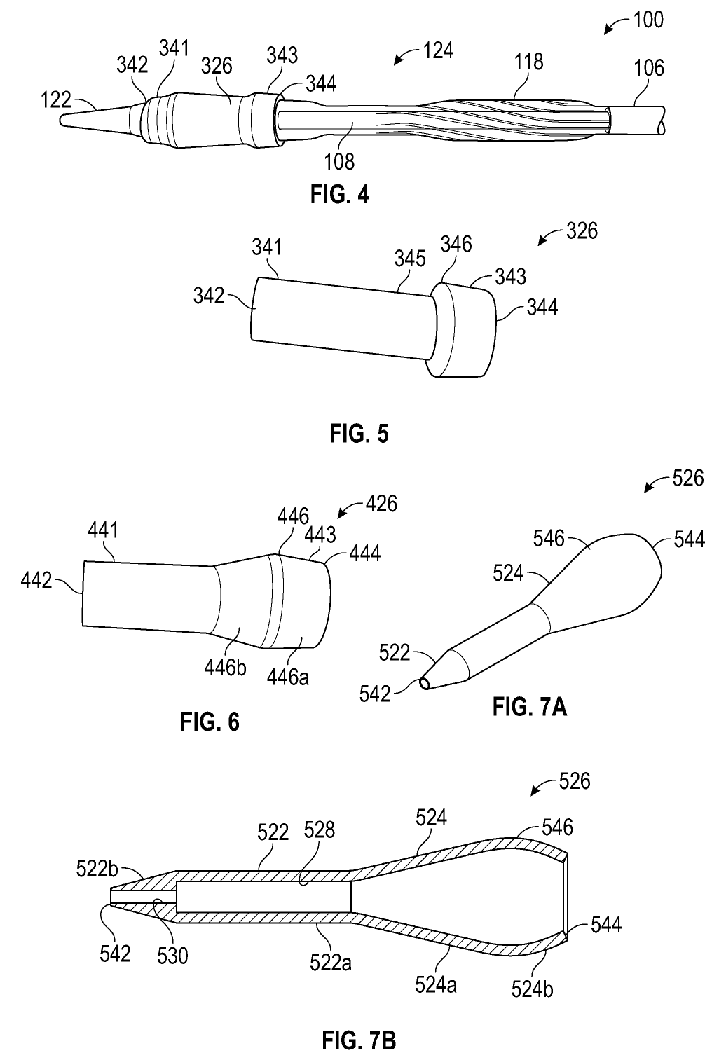

Additional examples of expandable distal shoulders having different configurations of protuberances are shown in FIGS. 4-6. Similar reference numbers are used to indicate structures having similar functionality. The distal shoulders shown in FIGS. 4-6 can be made from the same materials as disclosed for the distal shoulder 226 and can function in the same way as the distal shoulder 226.

An expandable distal shoulder 326 is illustrated in FIGS. 4-5 and an expandable distal shoulder 426 is illustrated in FIG. 6. As shown in FIG. 4, a distal portion 341 of the expandable distal shoulder 326 is positioned on and secured to (for example, with an adhesive) an outer surface of the nose cone 122 and a proximal portion 343 extends partially over the balloon 118 of the delivery apparatus 100, such that the expandable distal shoulder 326 is arranged adjacent to a distal side of the valve mounting portion 124. Similar to expandable distal shoulder 226, the expandable distal shoulder 326 includes a first end 342, a second end 344, and a protuberance 346 that is located on the proximal portion 343. Likewise, the expandable distal shoulder 426 includes a distal portion 441 defining a first end 442, a proximal portion 443 defining a second end 444, and a protuberance 446 that is located on the proximal portion 443. The expandable distal shoulders 326 and 426 are substantially similar to expandable distal shoulder 226, apart from the configuration of the protuberances, and can be formed from the materials described above in connection with the distal shoulder 226.

For example, as shown in FIGS. 5-6, the protuberance 346 comprises an enlarged, generally cylindrical shaped section, which extends from a relatively smaller cylindrical shaped section 345 of the proximal portion 343. The transition from the outer diameter of the smaller section 345 and to the maximum outer diameter of the protuberance 346 increases relatively quickly. The protuberance 446 comprises a generally cylindrical proximal portion 446a and a tapered, conical shaped distal portion 446b, such that the transition between the outer diameter at a location distal to the protuberance 446 and the maximum outer diameter of the protuberance 446 increases relatively gradually.

In some examples, an inner diameter of the expandable distal shoulders disclosed herein is constant in the axial direction from the distal end (for example, distal end 342) to the proximal end (for example, proximal end 344). The inner diameter of the expandable distal shoulders disclosed herein can be sized slightly smaller than an outer diameter of a proximal end of a nose cone (for example, nose cone 122). For example, as shown in FIG. 4, the distal portion 341 of the distal shoulder 326 can be stretched over the proximal end portion of the nose cone 122.

In some examples, the inner diameter of the expandable distal shoulders disclosed herein can be varied in the axial direction from the distal end to the proximal end. Referring to the expandable distal shoulder 426 illustrated in FIG. 6, for example, the expandable distal shoulder 426 can comprise a constant thickness, in some examples, such that the inner diameter of the expandable distal shoulder 426 is varied in the same manner along the axial direction as the outer diameter of the distal shoulder 426 (for example, generally increases from the distal end 442 to the proximal end 444). In examples having a varied inner diameter, the inner diameter at the distal end of the expandable distal shoulder can be sized approximately the same as or slightly smaller than an outer diameter of a proximal end of a nose cone (for example, nose cone 122), for example, to maintain engagement with the nose cone during advancement of the delivery apparatus 100 through a patient’s vasculature.

In some examples, the nose cone 122 can extend in the proximal direction over the distal end of the expandable distal shoulders disclosed herein, such that the distal end of the distal shoulder is positioned radially between the balloon 118 and the nose cone 122. For example, an interior surface of the nose cone 122 can be secured to an outer surface of an expandable distal shoulder.

In some examples, a nose cone and an expandable distal shoulder can be integrated together into a single unitary component. For example, as shown in FIGS. 7A-7B, an expandable distal shoulder 526 for a delivery apparatus (for example, delivery apparatus 100). The combination nose cone and shoulder 526 includes a nose cone portion 522 along the distal portion of the shoulder 526 and an enlarged, elastically expandable proximal portion 524 sized to extend over the distal portion of a balloon (for example, balloon 118). The nose cone portion 522 defines a first (or distal) end 542 of the expandable distal shoulder 526 and the proximal portion 524 defines a second (or proximal) end 544 of the expandable distal shoulder 526. In some examples, shoulder 526 can be formed as a unitary body (including the nose cone portion 522 and the proximal portion 524) by a molding process (for example, injection molding).

As best shown in FIG. 7B, the nose cone portion 522 can include a cylindrical proximal portion 522a and a tapered distal portion 522b. The proximal portion 522a can include a cavity or lumen 528 sized to receive an inner shaft 108 of a delivery apparatus (for example, delivery apparatus 100). The outer surface of the inner shaft 108 can be bonded (such as with an adhesive) to the inner surface of the lumen 528 so as to secure the shoulder 526 to the shaft 108. The distal portion 522b can include a respective lumen 530 that is in communication with a guidewire lumen of the shaft 108 such that a guidewire can extend through the shaft 108 and the lumen 530.

The proximal portion 524 of the shoulder 526 in the illustrated example forms a generally bell-shaped protuberance that includes a tapered distal portion 524a that increases in diameter from the proximal nose cone portion 522a to a tapered proximal portion 524b that decreases in diameter from the distal portion 524a to the proximal end 544 of the shoulder 526.

The distal shoulder 526 can be made from the same materials as disclosed for the distal shoulder 226 and can function in the same way as the distal shoulder 226. For example, when the balloon 118 is inflated to expand the prosthetic valve 150, the proximal portion 524 can radially expand with the balloon from a radially collapsed state to a radially expanded state. When the balloon is deflated, the proximal portion 524 can revert back to its radially collapsed state under its own resiliency. Moreover, the maximum outer diameter of the protuberance 546 can be greater than the outer diameter of the radially crimped prosthetic valve 150 to protect the leading edge of the prosthetic valve from contacting an inner surface of an introducer sheath and/or the surrounding anatomy.

FIGS. 8 and 9 illustrate the delivery apparatus 100 having an example of an elastically expandable distal shoulder 626 and an example of an expandable proximal shoulder 228 that are positioned on opposite sides of the valve mounting portion 124 of the delivery apparatus 100, radially outwards of the balloon 118. Specifically, the distal shoulder 626 has an undulating, curved hourglass shaped outer profile and is otherwise similar to the external distal shoulders of FIGS. 3-7 described above. For example, the distal shoulder 626 is connected to the nose cone 122 and defines an enlarged shoulder at a distal end of the valve mounting portion 124, such that the valve 150 is prevented from moving in a distal direction relative to the distal shoulder 626 when the valve 150 is crimped to the delivery apparatus 100. As shown, the distal shoulder 626 can have a distal portion a distal portion 626a that flares or increases in diameter from the nose cone 122 to an intermediate neck portion 626b that has a concave curvature that tapers or decreases in diameter than flares or increases in diameter to a proximal portion 626c, which has a convex curvature that defines a maximum diameter of the shoulder and then tapers slightly to the proximal end of the shoulder 626. Similar to the distal shoulders described above, the distal shoulder 626 also functions to protect the leading edge of the prosthetic valve 150 against contacting surrounding anatomy and an inner surface of an introducer sheath.

As shown, the delivery apparatus 100 of FIGS. 8-9 includes an expandable proximal shoulder 228, in lieu of the proximal shoulder (enlarged tip portion) 128 of the outer shaft 104. Thus, in example of FIGS. 8-9, the delivery apparatus 100 can have an outer shaft 104 (not shown in FIGS. 8-9), but the outer shaft need not have an enlarged tip portion 128 forming the proximal shoulder. Similar to the tip portion 128, the expandable proximal shoulder 228 can be configured to resist movement of the prosthetic valve 150 relative to the balloon 118 proximally, in the axial direction, for example, as the delivery apparatus and prosthetic heart valve are advanced through the patient’s vasculature and/or an introducer sheath. Unlike the tip portion 128, which is not configured to radially expand when the balloon 118 radially expands, the expandable proximal shoulder 228 is configured to radially expand when the balloon 118 is expanded. As shown, the proximal shoulder 228 is positioned radially outside of the balloon 118, for example, on an outer surface of a proximal end portion of the balloon 118. In this example, the delivery apparatus 100 is shown with the distal shoulder 626 and the proximal shoulder 228. However, it should be appreciated that delivery apparatuses can include any combination of the valve positioning structures disclosed herein (for example, distal shoulders, proximal shoulders, etc.) positioned on opposite sides of the valve mounting portion 124 in the distal and/or proximal positions.

The expandable proximal shoulder 228 is connected to the intermediate shaft 106, such that the expandable proximal shoulder 228 is positioned radially outwards of the balloon 118. In some examples, a proximal portion of the balloon 118 can be coupled to the intermediate shaft 106, such that the balloon 118 is positioned radially between intermediate shaft 106 and an inner surface of the expandable proximal shoulder 228. In some examples, the balloon 118 can be coupled to an inner surface of the intermediate shaft 106. In some examples, the expandable proximal shoulder 228 can be connected to the outer shaft 104 (for example, an inner or outer surface of the outer shaft 104), rather than to the intermediate shaft 106. In such examples when the proximal shoulder 228 is connected to the outer shaft 104, the proximal shoulder 228 functions as an enlarged tip portion of the shaft 104 and can be used for off-balloon delivery to apply a distally directed force to the prosthetic valve 150 for moving the prosthetic valve onto the valve mounting portion 124 once inside the patient’s vasculature.

As shown in FIGS. 10-11, the expandable proximal shoulder 228 includes a first (or inner) member 248 and a second (or outer) member 250. Both the inner member 248 and the outer member 250 are configured to expand in the radial direction, for example, upon radial expansion of the balloon 118 when the balloon is inflated. Specifically, as the balloon 118 is radially expanded, the balloon 118 exerts an expansion force against the inner member 248 and/or the outer member 250, which results in radial expansion of the expandable proximal shoulder 228 (for example, radial expansion of a portion of the expandable proximal shoulder 228 that is positioned around the balloon 118). When the balloon 118 is deflated prior to removal of the delivery apparatus 100 from the patient, the expandable proximal shoulder 228 is configured to contact to its unexpanded state, shown in FIG. 10.

As best shown in FIG. 11, the inner member 248, also referred to herein as a pronged member, includes a plurality of longitudinally extending prongs, or legs, 252 extending from a base 254. In some examples, the base 254 is connected to the intermediate shaft 106 (for example, to an outer surface of the intermediate shaft 106, to an inner surface of the intermediate shaft 106, etc.). Each of the prongs 252 extend in an axial direction from the base 254, for example, in a direction from the intermediate shaft 106 towards the balloon 118. As shown, the base 254 is generally annular and the prongs 252 are spaced apart from each other around the base 254 in a circumferential direction. In some examples, as depicted, each of the radially-facing inner surfaces and the radially-facing outer surfaces of the prongs 252 are curved, for example, such that the inner member 248 generally has an annular inner surface and an annular outer surface. The inner surfaces of the prongs 252 extend over and can lie against the outer surface of the proximal end portion of the balloon. The prongs 252 can flex or pivot relative to the base 254 and expand in the radially direction from a radially collapsed state (FIGS. 10 and 11) to a radially expanded state under the force of a balloon being inflated.

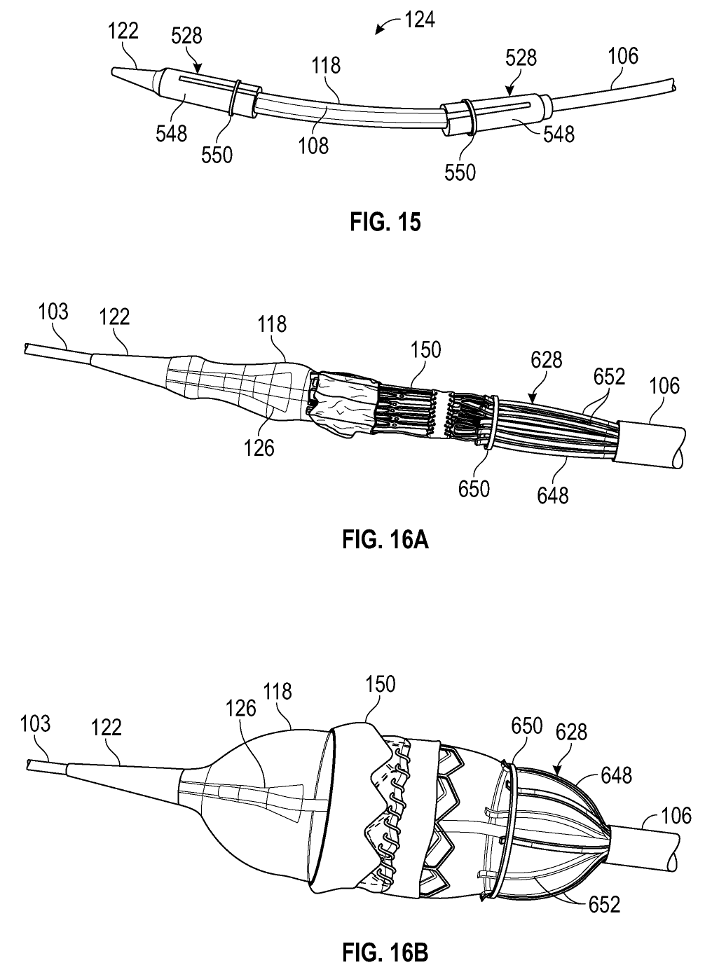

As shown, the inner member 248 includes eight prongs 252, although in other examples, the inner member 248 can include more than or less than eight prongs 252. For example, the pronged member 348, 648 shown in FIG. 13 and in FIG. 16B, respectively, each include six prongs and the pronged member 548 of FIG. 15 includes two prongs.

The outer member 250 is positioned radially outwards of the inner member 248 and substantially covers or surrounds the inner member 248. Specifically, the outer member 250, also referred to herein as a “sleeve,” can, for example, function to enclose the prongs 252 of the inner member 248 and create a smoother outer surface for the proximal shoulder 228. In this way, the sleeve 250 can enable the proximal shoulder 228 to advance through the patient’s vasculature in a manner that minimizes resistance between the native anatomy and the proximal shoulder 228.

The outer member 250 comprises an elastomeric and/or resilient material that is configured to expand upon inflation of the balloon 118 and contract when the balloon 118 is deflated. In some examples, the outer member 250 can comprise any of the materials listed herein for the expandable distal shoulders (for example, shoulder 226). Due to the resilient nature of the outer member 250, the outer member 250 assists in radially collapsing the prongs 252 from an expanded position to the radially collapsed position shown in FIGS. 8-10 under the resiliency of the outer member when the balloon is deflated. In this manner, the outer member 250 functions as a biasing member that exerts a biasing force against the prongs 252 that urges the prongs to move back to the radially collapsed state when the balloon is deflated.

As shown, the proximal shoulder 228 includes a first (or distal) end 256 and a second (or proximal) end 258. In the illustrated example, the base 254 is located at the second end 258 of the proximal shoulder 228 and the prongs 252 extend from the base 254 towards the first end 256 of the proximal shoulder 228. When the proximal shoulder 228 is coupled to the delivery apparatus 100, the second end 258 can be coupled to the intermediate shaft 106. In this configuration, the first end 256 and at least a portion of the prongs 252 can extend over a portion of the balloon 118. In this way, the first end 256 is positioned adjacent to the proximal end of the valve mounting portion 124, such that the first end 256 is adjacent to or in contact with the prosthetic valve 150, when the prosthetic valve 150 is mounted on the delivery apparatus 100. In this manner, the first end 256 of the expandable proximal shoulder 228 is configured to resist movement of the prosthetic valve 150 relative to the balloon 118 proximally, in the axial direction, for example, during advancement of the delivery apparatus 100 through the patient’s vasculature and/or an introducer sheath as well as when as the balloon 118 is expanded.

When the balloon 118 is expanded, the prongs 252 of the inner member 248 expand in the radial direction. Specifically, as the balloon 118 is radially expanded, the balloon 118 exerts an expansion force against the prongs 252 of the inner member 248 and the prongs 252 flex radially outwards relative to the base 254. At the same time, the outer member 250 expands radially and circumferentially from the expansion force of the balloon 118. When the balloon 118 is deflated prior to removal of the delivery apparatus 100 from the patient, the prongs 252 return to their original, unflexed and radially collapsed configuration shown in FIG. 11 under the resiliency of the outer member 250.

In some examples, the inner member 248 comprises a material that is generally stiffer and/or more rigid than the outer member 250. As such, during advancement of the delivery apparatus through the patient’s vasculature, the inner member 248 maintains the structural integrity of the proximal shoulder 228 and counteracts axially-directed forces applied by the valve 150. As such, in some examples, the inner member 248 functions as a support structure that can push the valve 150 through the patient’s vasculature. In some examples, the inner member 248 is made of a relatively rigid and/or non-elastic polymers, such as nylon or high-density polyethylene, while the outer member 250 can be made of any of various elastomeric materials, including any of those described above for the distal shoulder 226.

As shown in FIG. 10, the outer member 250 optionally can include a lip portion 260 that extends in the axial direction beyond the distal end of the inner member 248 and extends radially inwards over the distal ends of the prongs 252. In this way, the lip portion 260 of the outer member 250 is configured to wrap around the distal end of the inner member 248, such that the valve 150 is positioned adjacent to and/or contacts the outer member 250 of the proximal shoulder 228 when crimped on the valve mounting portion 124 of the delivery apparatus 100. The outer member 250 optionally can include a similar lip portion 260 that extends axially beyond and radially inward over a proximal end of the inner member 248.

In some examples, the outer member 250 can include a lip portion 260 at only one end of the outer member 250. For example, the outer member 250 can include one lip portion 260 that extends in the axial direction beyond adjacent end of the inner member 248 and extends radially inwards at the adjacent end (for example, at the distal end 256) of the proximal shoulder 228.

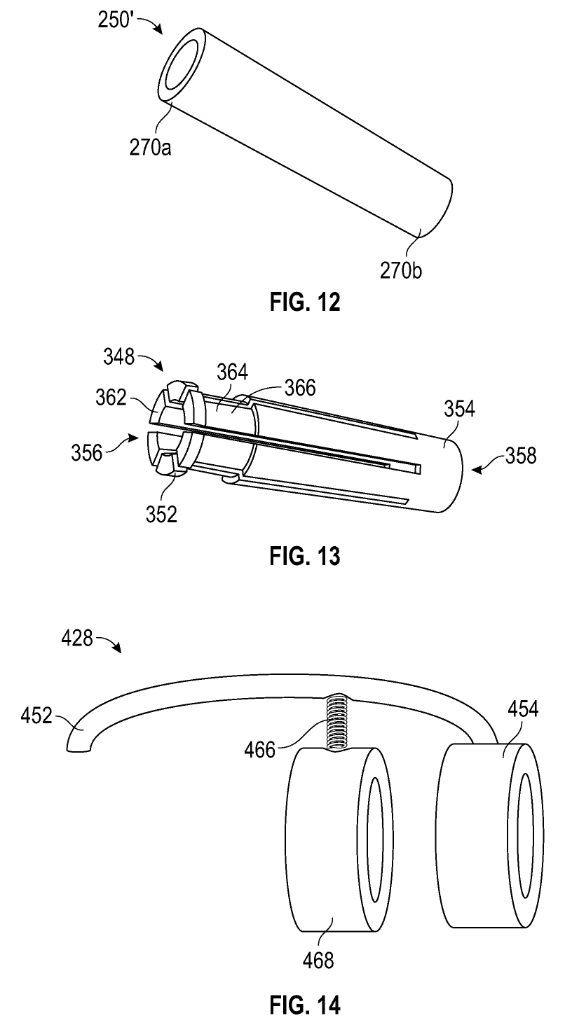

In some examples, the outer member does not include a lip portion at either end. For example, FIG. 12 illustrates an example of an outer member 250’ that can be used with the inner member 248, in lieu of outer member 250. The outer member 250’ can have a cylindrical shape having a constant diameter along its length as shown. The outer member 250’ can be disposed over the inner member 248, such that the outer member 250’ surrounds the outer surfaces of the prongs 252. The outer member 250’ is configured to help radially collapse the prongs 252 of the inner member 248 from the radially expanded position upon inflation of the balloon to the radially collapsed position when the balloon is deflated.

In some examples, the outer member 250’ optionally can be stretched over the inner 248, meaning that the inner diameter of the outer member 250’ is slightly smaller than a maximum outer diameter of the inner member 348 when the outer member 250’ is in a relaxed, non-deformed state. In this manner, when outer member 250’ is placed over the inner member 248, the outer member 250’ is slightly expanded or stretched radially and applies a small compression force against the prongs 252 when the prongs 252 are radially collapsed and the balloon is deflated. This can be referred to as a “pre-stretched” state of the outer member 250’.

In some examples, the outer member 250’ has an axial length that is greater than the length of the inner member 248. In these examples, the outer member 250’ can surround the outer surfaces of the prongs 252 and the base 254 and opposing end portions 270a, 270b of the outer member 250’ can extend axially beyond the adjacent ends of the inner member. If the outer member 250’ is held in a pre-stretched state over the inner member 248, the opposing end portions 270a, 270b (which are not in contact with the outer surface of the inner member 248), can wrap at least partially around the opposing ends of the inner member 248. In this manner, the proximal end of the valve 150 can contact an adjacent end of the outer member 250’ rather than the inner member 248 when mounted on the valve mounting portion 124 of the delivery apparatus 100.

In some examples, the outer member 250’ has an axial length that is less than or equal to a length of the inner member that it surrounds (for example, inner member 248). In such examples, the outer member 250’ does not wrap around the opposing ends of the inner member 248, such that the valve 150 is configured to contact the inner member 248 when mounted on the valve mounting portion 124 of the delivery apparatus 100.

The expandable proximal shoulders disclosed herein (for example, expandable proximal shoulder 228, etc.) can also be referred to more generally as valve positioning structures. In some examples, the proximal shoulders can be positioned at either a distal or proximal end of the valve mounting portion 124. For example, rather than being positioned adjacent to the proximal end of the valve mounting portion 124 (as shown in FIGS. 8-9), the expandable valve positioning structure 228 can instead be positioned adjacent to the distal end of the valve mounting portion 124. In this positioning, the second end 258 can be connected to the nose cone 122 and the first end 256 can be oriented towards the leading end of the prosthetic valve 150, such that the expandable valve positioning structure 226 functions as a distal shoulder. In such examples, the positioning structure 226 extends over a distal end portion of the balloon 118 and radially expands and collapses when the balloon is inflated and deflated, respectively.

Similarly, the distal shoulders disclosed herein can also be referred to more generally as valve positioning structures. In some examples, the distal shoulders can be positioned at either a distal or proximal end of the valve mounting portion 124. Using the distal shoulder 226 of FIGS. 3A-3B as an example, the expandable valve positioning structure 226 can instead be positioned adjacent to the proximal end of the valve mounting portion 124, rather than being positioned adjacent to the distal end of the valve mounting portion 124 as is shown in FIGS. 3A-3B. In this positioning, the first end 242 can be connected to the intermediate shaft 106 or the main shaft 104 (for example, adjacent the proximal end of the valve mounting portion 124) and the second end 244 is oriented towards the proximal end of the prosthetic valve 150, such that the expandable valve positioning structure 226 functions as a proximal shoulder. For example, at least a portion of the expandable valve positioning structure 226 can comprise a material that is sufficiently stiff and/or rigid, such that during advancement of the delivery apparatus through the patient’s vasculature, the expandable valve positioning structure 226 can maintain its structural integrity and counteract axially-directed forces applied by the valve 150 against the second end 244 of the valve positioning structure 226, while also being sufficiently flexible and resilient to expand and collapse upon inflation and deflation of the balloon, respectively. As such, in some examples, the valve positioning structure 226 can function as a support structure that can push the valve 150 through the patient’s vasculature. When connected to the outer shaft 104, the positioning structure 226 is an enlarged tip portion of the outer shaft and can be used to move the prosthetic valve from its initial offset location to the valve mounting portion 124 of the balloon during an off-balloon delivery procedure.

FIG. 13 illustrates an example of an inner (or pronged) member 348 for an expandable valve positioning structure. Similar to inner member 248, the inner member 348 includes a plurality of prongs 352 extending from a base 354. The inner member 348 includes a first (or distal) end 356 and a second (or proximal) end 358. Like prongs 252, the prongs 352 are configured to flex radially outwards relative to the base 354 when the balloon 118 is inflated. When the balloon 118 is deflated prior to removal of the delivery apparatus 100 from the patient, the prongs 352 return to their original, unflexed and radially collapsed configuration shown in FIG. 13.

The base 354 is generally annular and includes a constant outer diameter (for example, such that the base 354 is generally cylindrical). In some examples, as depicted, a portion of the prongs 352 have the same outer diameter as the base 354, for example, at the location where the prongs 352 extend from the base 354.

As shown, the inner member 348 includes six prongs 352. In some examples, the inner member 348 can include a greater or fewer number of prongs 352. Each prong 352 includes a flange 362 at the first end 356 of the inner member 348. In some examples, the flanges 362 of the prongs 352 are configured to push against the valve 150, for example, when the valve 150 is mounted on the valve mounting portion 124 of the delivery apparatus 100. In other words, the flanges 362 can define a surface that can contact the valve 150 to maintain the position of the valve 150 relative to the delivery apparatus 100 during advancement of the delivery apparatus 100 through the patient’s vasculature.

Adjacent to the flange 362, each prong 352 can include a channel or recessed portion 364 extending in a circumferential direction. Each channel portion 364 has a length in the axial direction that is less than a length of the prong 352. The channel portion 364 has an outer diameter that is less than an outer diameter of the flange 362 and that is less than an outer diameter of the base 354. In this way, the channel portions 364 of the prongs 352 collectively define a channel 366 is recessed relative to the flanges 362 and the base 354.

In some examples, an outer member, similar to outer member 250’, can be positioned within the channel defined by the channel portions 364 of the prongs 352. Specifically, the outer member 250’ can be modified to have an axial length that is less than or equal to an axial length of the channel portions 364 and therefore need not extend over the prongs 362 except for portions of the prongs forming the channel. In these examples, the outer member 250’, also referred to as a band formed from an elastomeric and/or resilient material, can expand radially to allow the prongs 352 to flex radially outwards relative to the base 354 when the balloon is inflated and, due to the resiliency of the band 250’, the band 250’ can assist in collapsing the prongs 352 from the flexed, expanded position to the radially collapsed position shown in FIG. 13 under a radial compression force applied by the band 250’ on the prongs 352. In this manner, the band 250’ functions as a biasing element for the prongs 352.

In some examples, an outer member having an axial length greater than the axial length of the channel portions 364 (for example, equal to or greater than an axial length of the inner member 348) can be positioned around the inner member 348.

In some examples, to help collapse the prongs from the radially expanded position to the radially collapsed position, pronged member can include a retraction or biasing member in the form of a spring, to return the prongs to the radially collapsed position in lieu of an elastomeric outer member (for example, outer members 250, 250’). For example, as shown in FIG. 14, a pronged member 428 includes a base 454 and a prong 452 extending from the base 454. While only one prong 452 is shown in FIG. 14, it should be appreciated that the pronged member 428 can include additional prongs 452.

In some examples, as shown in FIG. 14, the pronged member 428 includes a tension spring 466 coupled to a spring base 468 and the prong 452 at opposing ends of the spring. The spring 466 can be configured to apply a biasing force to the prong 452 to urge the prong 452 from the flexed, expanded position to the radially collapsed position when the balloon is deflated. As shown, the spring 466 is positioned radially between the prong 452 and the spring base 468. In the illustrated example, the prong 452 has a generally curved profile along its axial length, for example, to accommodate the spring 466 in a position that is radially between the prong 452 and the spring base 468. In some examples, the prong 452 can have other profiles such as a generally straight profile, a profile that includes a flange (for example, similar to flange 362 extending radially outwards from the rest of the prong, a flange extending radially inwards from the rest of the prong, etc.). A spring 466 can be provided for each prong 452 of the pronged member 428. One end of each spring 466 can be connected to the spring base 468 and the opposite end of each spring 466 can be connected to a respective prong 452.

The spring 466 is coupled to the prong 452 at a location that is spaced away from the fixed end of the prong 452 (for example, the end of the prong 452 that is coupled to the base 454). The distance between the location and the fixed end of the prong 452 can be adjusted based on the stiffness of the spring, for example.

As shown, the spring base 468 can be separate from the base 454 and spaced apart in the axial direction. In some examples, the spring(s) 466 can be attached to the base 454, such that the spring base 468 can be omitted. In some examples, the base 454 and the spring base 468 can both be coupled to the same structure or component of a delivery apparatus. For example, when the pronged member 428 is used as a distal positioning structure, the base 454 and the spring base 468 can be coupled to the nose cone 122. In some examples, when the pronged member 428 is used as a proximal positioning structure, the base 454 and the spring base 468 can be coupled to the intermediate shaft 106 or the outer shaft 104.

In some examples, the spring(s) 466 can be included in addition to an outer member, such as those described herein (for example, band 250’). In this way, the spring(s) 466 can assist in urging the prongs 452 from the flexed, expanded position to the radially collapsed position, in addition to the outer member. In some examples, each of the prongs 452 can include a flange (for example, similar to flange 362) and a channel portion, such that an outer member can be positioned within a channel defined by the channel portions of the flanges.

FIG. 15 illustrates an example of a valve mounting portion 124 of the delivery apparatus 100. In some examples, as shown, the same positioning structure (for example, valve positioning structure 528) can be used as a distal shoulder and as a proximal shoulder. For example, one valve positioning structure 528 is connected to the nose cone 122 at the distal end of the valve mounting portion 124 and extends over a distal portion of the balloon and another valve positioning structure 528 is coupled to the intermediate shaft 106 (or the outer shaft 104) at the proximal end of the valve mounting portion 124 and extends over a proximal portion of the balloon. Each valve positioning structure 528 includes a pronged member 548 and an elastomeric outer member 550 extending around the prongs of the pronged member 548. The pronged member 548 and outer member 550 are similar to and function in the same way as the pronged members and outer members described herein. Thus, when the balloon 118 is inflated, the proximal and distal prong members 548 radially expand under the expansion force of the balloon and radially collapsed under the resiliency of the outer members 550 when the balloon is deflated. In some examples, as shown in FIGS. 8-9, for example, different structures can be used as a distal shoulder and as a proximal shoulder (for example, expandable valve positioning structure 626 and expandable valve positioning structure 228, respectively).

As shown in FIGS. 16A-16B, the delivery apparatus 100 can have both an internal, non-expandable shoulder and an external, expandable shoulder positioned at opposite ends of the valve mounting portion 124. For example, as shown, the delivery apparatus can include the internal distal shoulder 126, which is non-expandable, positioned internal to the balloon 118 and an expandable, external proximal shoulder 628 is positioned external to the balloon 118. The shoulder 628 includes a pronged member 648 (for example, similar to the pronged members described herein) and an outer member 650 in the form of an elastomeric annular band or sleeve (for example, similar to outer members described herein). The balloon 118 is shown transparently for purposes of illustration. The pronged member 648 includes a plurality of prongs 652 that extend over a proximal portion of the balloon 118. The proximal ends of the prongs 652 can be connected directly to a distal end of the shaft 106 or to a base (similar to 254), which in turn is connected to the distal end of the shaft 106 (or shaft 104).

FIG. 16A shows the balloon 118 in an uninflated state with the prongs 652 in a radially collapsed state. In this state, the distal ends of the prongs 652 can contact the proximal end of the prosthetic valve 150 to resist movement of the prosthetic valve in the proximal direction as the delivery apparatus is advanced through an introducer sheath and the patient’s vasculature. FIG. 16B shows the balloon in the inflated state, the prosthetic valve 150 in the radially expanded state, and the prongs 652 in the radially expanded state. When the balloon 118 is deflated, the prongs 652 can revert back to the radially collapsed state (FIG. 16A) under the resiliency of the outer member 650.

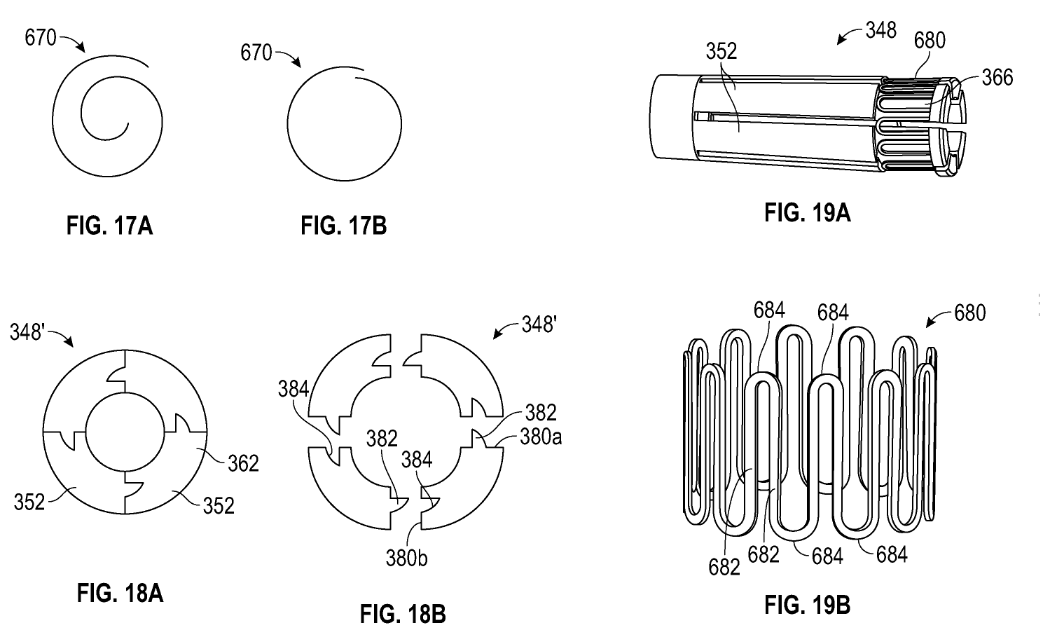

FIGS. 17A-17B illustrate a biasing member 670 that can be used with a pronged member, such as pronged member 348. The biasing member 670 comprises an expandable coil shaped to fit within the channel 366 of the pronged member 348. The biasing member can be coiled wire or coiled band made of a shape memory material, such as Nitinol. The biasing member can be shaped set in a radially collapsed state (FIG. 17A) and can expand radially to a radially expanded state (FIG. 17B) under the expansion force of the prongs 352, when they are expanded by a balloon 118 being inflated. When the balloon is deflated, the biasing member 670 reverts back to its radially collapsed state under its own resiliency, thereby urging the prongs 352 back to their radially collapsed state.

In the radially collapsed state, the biasing member 670 can have one or more radially overlapping windings. In some examples, the biasing member 670 can have one or more helically wrapped windings. In the figures, the windings are depicted with gaps between adjacent windings for purposes of illustration. It should be understood that biasing member 670 desirably is configured with adjacent windings in contact with each other.

FIGS. 18A-18B are end views of a pronged member 348’, which is similar to pronged member 348 except for the following modifications. The pronged member 348’ is depicted as having four prongs 352, but it can include a greater or fewer number of prongs (for example, six prongs 352, as depicted in FIG. 13). As shown, each prong 352 includes opposing, first and second longitudinally extending sides 380a, 380b, respectively. The first side 380a can be formed with a circumferentially extending projection 382 and the second side 380b can be formed with a recess or notch 384 shaped to receive a projection 382 of an adjacent prong 352. Each recess 384 can have a shape corresponding to the shape of an adjacent projection 382.

In the radially collapsed state of the prongs 352 (FIG. 18A), each projection 382 nests within an adjacent recess 384 of an adjacent prong 352. In this manner, the projections 382 and recesses 384 function as interlocking features that can maintain parallel alignment of the prongs 352 in the radially collapsed state as the delivery apparatus is advanced through a patient’s vasculature. Upon inflation of the balloon 118, the prongs 352 can expand radially as previously describing while the projections 382 move out of the notches 384 (FIG. 18B). When the balloon is deflated, the prongs 352 move back to the radially collapsed state and the projections 382 move back into their respective notches 384. In some examples, in lieu of or in addition to the projections 382 and recesses 384, the prongs 352 can include other interlocking features, such as features that form a snap fit engagement between adjacent prongs.

The projections 382 and the recesses 384 extend partially or entirely along the length of the first and second sides 380a, 380b of the prongs 352. In some examples, the projections 382 and the recesses 384 extend only along the free end portions of the prongs (for example, only along the flange portion 362).

The pronged member 348’ can be used with any of the outer members or biasing members disclosed herein for providing a biasing force that urges the prongs to move back to the radially collapsed state after being expanded. Moreover, the pronged member 348’can be used as a proximal shoulder and/or a distal shoulder in a delivery apparatus (for example, delivery apparatus 100).

FIGS. 19A and 19B show a biasing member 680 that can be used with the pronged member 348 (or any of the pronged members disclosed herein). The biasing member 680 can be sized to fit within the recess 366 of the pronged member. The biasing member 680 comprises an expandable annular body in the form of a ring or band comprising a plurality of circumferentially spaced longitudinal struts 682 connected to each other by connecting portions 684 in a serpentine pattern. The biasing member 680 desirably is a closed ring or band, meaning that it does not have any free ends that can overlap each other in the radial direction to form windings (unlike the outer member 670).

The biasing member 680 can made of a shape memory material, such as Nitinol. The biasing member can be shaped set in a radially collapsed state (FIG. 19A) and can expand radially to a radially expanded state under the expansion force of the prongs 352, when they are expanded by a balloon 118 being inflated. When the balloon is deflated, the biasing member 680 reverts back to its radially collapsed state under its own resiliency, thereby urging the prongs 352 back to their radially collapsed state.

The biasing member 680 can be formed by laser cutting tube of material (for example, a Nitinol tube). The biasing member 680 can have other shapes than that shown in FIGS. 19A-19B. For example, the biasing member 680 can have a plurality of circumferentially extending rows of angled struts arranged to form a plurality of cells, similar to a stent or a frame for a prosthetic heart valve.

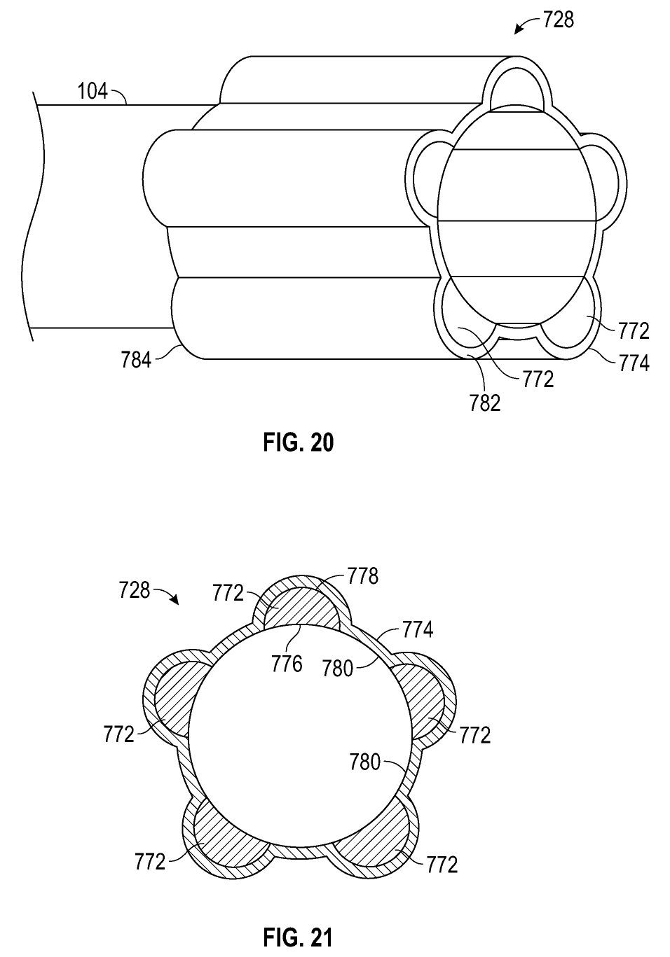

FIGS. 20-21 illustrate an example of a valve positioning structure 728 in the form of an enlarged tip portion for a catheter shaft that can function as an expandable proximal shoulder. Specifically, the valve positioning structure 728 is configured to radially expand when a balloon (for example, balloon 118) is expanded, and is configured to be positioned radially outside of the balloon, for example, on an outer surface of the balloon. In some examples, the valve positioning structure 728 is connected to the distal end portion of the outer shaft 104 of the delivery apparatus 100 (in place of tip portion 128). To connect the valve positioning structure 728 to the shaft 104, the distal end portion of the shaft 104 can extend partially into valve positioning structure 728 and the outer surface of the shaft 104 can be bonded to the inner surface of the valve positioning structure, such as with an adhesive.

The valve positioning structure 728 includes first (or inner) members 772 and a second (or outer) member 774. Both the inner members 772 and the outer member 774 are configured to expand in the radial direction, for example, upon radial expansion of a balloon of a delivery apparatus. Specifically, as the balloon is radially expanded, the balloon exerts a force against the inner members 772 and/or the outer member 774, which results in radial expansion of the valve positioning structure 728 (for example, radial expansion of a portion of the valve positioning structure 728 that is positioned around the balloon). When the balloon is deflated prior to removal of the delivery apparatus 100 from the patient, the valve positioning structure 728 is configured to contract to its unexpanded state, shown in FIG. 20.

The inner members 772, also referred to herein as rods or stiffening members, extend in an axial direction along a length of the valve positioning structure 728. As shown, the valve positioning structure 728 comprises has an annular shape and the rods 772 are spaced apart from each other around the valve positioning structure 728 in a circumferential direction. When the valve positioning structure 728 is radially expanded, for example, by a balloon of a delivery apparatus, the spacing between each adjacent rod 772 increases in the circumferential direction.

In some examples, as depicted, each of the radial inner surfaces 776 and the radial outer surfaces 778 of the rods 772 are curved. The inner surfaces 776 of the rods 772 and angular sections 780 of the inner surface of the outer member 774 collectively define a cylindrical inner surface of the structure 728 that can mate with the outer surface of the shaft. As shown, the valve positioning structure 728 includes five rods 772, although in some examples, the valve positioning structure 728 can include a greater or fewer number of rods 772.

The outer member 774 comprises a material that is configured to elastically expand (for example, as the balloon is inflated) and contract (for example, as the balloon is deflated). Thus, the outer member 774 can be referred to as an elastomeric outer layer. In some examples, the outer member 774 can comprise any of the materials listed herein for the elastically expandable distal shoulders (for example, shoulder 226) and/or outer member 250, including, but not limited to as natural rubber or any of various synthetic elastomers described above in connection with other disclosed embodiments, including but not limited to, as polyurethane (for example, Tecoflex™).

When the balloon is expanded, the rods 772 and the outer member 774 move outwards relative to a central longitudinal axis of the delivery apparatus in the radial direction. Specifically, as the balloon is radially expanded, the balloon exerts a force against the rods 772 and at least a portion of the outer member 774 and the structure 728 assumes a radially expanded state around the outer surface of the balloon. For example, when the structure 728 extends over a proximal end portion of the balloon, the structure 728 can expand to assume a generally conical or frustoconical shape that tapers in diameter from a distal end 782 to a proximal end 784 of the structure 728. When the balloon is deflated prior to removal of the delivery apparatus from the patient, the rods 772 and outer member 774 return to their original, unexpanded state shown in FIG. 20 under the resiliency of the outer member 774.

In some examples, the rods 772 comprise a material that is generally stiffer and/or more rigid than the outer member 774. For example, the rods 772 can be made of a relatively rigid and/or non-elastic polymer, such as nylon or high-density polyethylene. In some examples, the rods 772 can be made of a metal, such as any of the materials described above for the valve frame 12. As such, during advancement of the delivery apparatus through the patient’s vasculature, the rods 772 maintain the structural integrity of the valve positioning structure 728 and counteract axially-directed forces applied by the valve 150. Thus, in some examples, the rods 772 function as a support structure that can push the valve 150 through the patient’s vasculature.

When delivering a prosthetic valve 150 in an on-balloon procedure, the prosthetic valve 150 can be radially crimped on the valve mounting portion 124 of the balloon 118 and the structure 728 can be positioned over the proximal end portion of the balloon 118 such that the distal end 782 is adjacent or abuts a proximal end 150p of the prosthetic valve 150. In this manner, the structure 728 can maintain the position of the prosthetic valve on the balloon as the prosthetic valve and the delivery apparatus are inserted through an introducer sheath and the patient’s vasculature. When delivering a prosthetic valve in an off-balloon procedure, the prosthetic valve 150 can be initially crimped in an offset position (such as shown in FIG. 2A), the structure 728 can be used to move (for example, push) the prosthetic valve 150 from the offset position to the valve mounting portion 124 of the balloon, after which the structure 728 can be used to assist in maintain the position of the prosthetic valve until it is deployed, as previously described.

The outer member 774 is positioned radially outwards of the rods 772 and substantially covers or surrounds the rods 772. In some examples, the outer member 774 and the rods 772 can be coextruded, such that the outer member 774 and rods 772 are extruded together to produce a multilayer structure. However, other manufacturing techniques can be used to form the structure 728. For example, the outer member 774 and the rods 772 can be separately formed (such as by molding or extruding each component separately) and then connecting the rods 772 to the inner surface of the outer member 774, such as with an adhesive or welding. The outer member 774 desirably completely covers outer surfaces of the rods 772 to enable the valve positioning structure 728 to advance through the patient’s vasculature in a manner that minimizes resistance between the native anatomy and the rods 772 of the valve positioning structure 728.

A delivery apparatus 100 that includes the structure 728 connected to the distal end portion of the shaft 124 optionally can include any of the internal or external distal shoulders disclosed herein. Moreover, the structure 728 can be used at other locations on the delivery apparatus 100. For example, the structure can be connected to the distal end portion of the shaft 106 for use as a proximal shoulder. In some examples, in some examples, the structure 728 can be mounted on the distal end portion of the balloon 118 for use as a distal shoulder. In such examples, a distal portion of the structure 728 can be connected to the nose cone 122 (for example, bonded to the outer surface of the nose cone) and a proximal portion of the structure 728 can extend over the distal portion of the balloon.

Any of the valve positioning structures or shoulders disclosed herein can include a lubricious and/or hydrophilic outer coating to reduce sliding friction with an introducer sheath and/or the patient’s vasculature as the delivery apparatus is advanced to the implantation site. A coating can comprise polyvinylpyrrolidone (PVP). The coating can be formed by, for example, spraying the coating material onto the valve positioning structure, or dip coating the valve positioning structure into liquified coating material. In some examples, a dip coating process can comprise immersing the valve positioning structure in a solution comprising a polymer, such as PVP, dissolved in a solvent, such as dimethyl acetamide (DMAC), removing the valve positioning structure from the solution and allowing it to cure.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (for example, by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient’s vasculature. Moreover, the disclosed delivery approaches are not intended to be limited. Any of the prosthetic valves disclosed herein can be implanted using any of various delivery procedures and delivery devices known in the art.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A delivery apparatus for delivering a prosthetic valve through vasculature of a patient, the delivery apparatus comprising: a handle; a shaft coupled to the handle and a balloon coupled to a distal end portion of the shaft, wherein the balloon is configured to be inflated from a first, deflated state to a second, radially expanded, inflated state, wherein the balloon has a valve mounting portion for mounting the prosthetic valve in a radially compressed state; and a valve positioning structure extending at least partially over an exterior surface of the balloon and having a first end portion and a second end portion, wherein the valve positioning structure is configured to radially expand from a radially collapsed state to a radially expanded state when the balloon is inflated and then radially collapse from the radially expanded state to the radially collapsed state when the balloon is deflated.

Example 2. The delivery apparatus of any example herein, particularly example 1, wherein the valve positioning structure is positioned along a distal end portion of the balloon, and wherein the valve positioning structure is configured to limit distal movement of the prosthetic valve relative to the balloon.

Example 3. The delivery apparatus of any example herein, particularly examples 1 or 2, wherein the valve positioning structure comprises an integral nose cone that is connected to the distal end portion of the shaft distal to the balloon.

Example 4. The delivery apparatus of any example herein, particularly examples 1 or 2, further comprising a nose cone connected to the distal end portion of the shaft, wherein the first end portion of the valve positioning structure is connected to the nose cone.

Example 5. The delivery apparatus of any example herein, particularly example 4, wherein an inner surface of the first end portion of the valve positioning structure is bonded to an outer surface of the nose cone.

Example 6. The delivery apparatus of any example herein, particularly example 1, wherein the valve positioning structure is positioned along a proximal end portion of the balloon, and wherein the valve positioning is configured to limit proximal movement of the prosthetic valve relative to the balloon.

Example 7. The delivery apparatus of any example herein, particularly example 6, wherein the shaft comprises a first shaft, and the delivery apparatus further comprises a second shaft extending coaxially over the first shaft, and wherein the first end portion of the valve positioning structure is connected to a distal end portion of the second shaft.

Example 8. The delivery apparatus of any example herein, particularly example 7, wherein the second shaft is movable axially relative to the first shaft and the balloon, and the valve positioning structure is configured to move the prosthetic valve relative to the balloon from an offset position to the valve mounting portion of the balloon.

Example 9. The delivery apparatus of any example herein, particularly any one of examples 1-8, wherein a maximum outer diameter of the valve positioning structure is greater than an outer diameter of the prosthetic valve in the radially compressed state.

Example 10. The delivery apparatus of any example herein, particularly any one of examples 1-9, wherein the first end portion of the valve positioning structure has an outer diameter that is smaller than an outer diameter of the second end portion of the valve positioning structure.

Example 11. The delivery apparatus of any example herein, particularly example 10, wherein the valve positioning structure comprises a protuberance positioned along the second end portion of the valve positioning structure.

Example 12. The delivery apparatus of any example herein, particularly example 11, wherein the protuberance defines a maximum outer diameter of the valve positioning structure.

Example 13. The delivery apparatus of any example herein, particularly example 11 or example 12, wherein the protuberance extends around the circumference of the valve positioning structure.

Example 14. The delivery apparatus of any example herein, particularly any of examples 1-13, wherein the valve positioning structure has an hour-glass shape.

Example 15. The delivery apparatus of any example herein, particularly any of examples 1-14, wherein the valve positioning structure is elastically expandable and collapsible upon inflation and deflation of the balloon, respectively.

Example 16. The delivery apparatus of any example herein, particularly any of examples 1-15, wherein the valve positioning structure stretches radially and circumferentially when the balloon is inflated and contracts radially and circumferentially when the balloon is deflated.

Example 17. The delivery apparatus of any example herein, particularly any of examples 1-16, wherein the valve positioning structure is made of an elastomer.

Example 18. The delivery apparatus of any example herein, particularly example 16, wherein the elastomer comprises polyurethane.

Example 19. The delivery apparatus of any example herein, particularly any one of examples 1-9, wherein the valve positioning structure comprises an inner member and an outer member positioned radially outwards of the inner member.

Example 20. The delivery apparatus of any example herein, particularly example 19, wherein the inner member comprises a base and a plurality of prongs extending axially from the base, wherein the prongs extend at least partially over the exterior of the balloon and are configured to transition from the radially collapsed state to the radially expanded state when the balloon is inflated, and wherein the outer member is configured to urge the prongs back to the radially collapsed state when the balloon is deflated.

Example 21. The delivery apparatus of any example herein, particularly example 20, wherein each prong comprises a channel portion extending in the circumferential direction, and wherein the outer member is positioned in the channel portions of the prongs.

Example 22. The delivery apparatus of any example herein, particularly example 20 or example 21, wherein each prong comprises a fixed end portion connected to the base and a free end portion opposite the fixed end portion.

Example 23. The delivery apparatus of any example herein, particularly any of examples 20-22, wherein the free end portion of each prong comprises a radially extending flange.

Example 24. The delivery apparatus of any example herein, particularly any one of examples 20-23, wherein the inner member comprises a material that is more rigid than the outer member.

Example 25. The delivery apparatus of any example herein, particularly any one of examples 20-24, wherein the outer member comprises an elastomeric band or sleeve.

Example 26. The delivery apparatus of any example herein, particularly example 25, wherein the outer member is made of an elastomer.

Example 27. The delivery apparatus of any example herein, particularly any of examples 20-24, wherein the outer member comprises a coiled member.

Example 28. The delivery apparatus of any example herein, particularly example 27, wherein the outer member comprises coiled wire or band.

Example 29. The delivery apparatus of any example herein, particularly any of examples 27-28, wherein the outer member is made of a shape memory material and is shaped set in a radially collapsed state.

Example 30. The delivery apparatus of any example herein, particularly example 29, wherein the shape memory material comprises Nitinol.

Example 31. The delivery apparatus of any example herein, particularly any of examples 20-24, wherein the outer member comprises an annular band made of Nitinol.

Example 32. The delivery apparatus of any example herein, particularly example 31, wherein the annular band comprises a plurality of axial struts having opposing ends, each of which is connected to an adjacent end of an adjacent strut by a connecting portion of the band.

Example 33. The delivery apparatus of any example herein, particularly any of examples 31-32, wherein the outer member is shape set in a radially collapsed state.

Example 34. The delivery apparatus of any example herein, particularly any of examples 31-33, wherein the outer member is a closed annular band.

Example 35. The delivery apparatus of any example herein, particularly any of examples 1-9, wherein the valve positioning structure comprises a base, a plurality of prongs extending axially from the base, and a plurality of biasing elements, wherein the prongs extend at least partially over the exterior of the balloon and are configured to transition from the radially collapsed state to the radially expanded state when the balloon is inflated, and wherein the biasing elements are configured to urge the prongs back to the radially collapsed state when the balloon is deflated.

Example 36. The delivery apparatus of any example herein, particularly example 35, wherein the biasing elements comprise springs.

Example 37. The delivery apparatus of any example herein, particularly example 36, wherein the springs comprise tension springs.

Example 38. The delivery apparatus of any example herein, particularly any of examples 36-37, wherein a first end of each spring is coupled to one of the prongs, wherein a second end of the spring is coupled to a spring base.

Example 39. The delivery apparatus of any example herein, particularly example 38, wherein the spring base is an extension of the base from which the prongs extend.

Example 40. The delivery apparatus of any example herein, particularly any of examples 1-9, wherein the valve positioning structure comprises a plurality of axially extending rods and an annular, elastomeric layer extending around the rods.

Example 41. The delivery apparatus of any example herein, particularly example 40, wherein the rods are made of a first material and the elastomeric layer is made of a second material, wherein the first material is stiffer than the second material.

Example 42. The delivery apparatus of any example herein, particularly any of examples 40-41, wherein the elastomeric layer and the rods comprise different materials that are coextruded together.

Example 43. The delivery apparatus of any example herein, particularly any of examples 40-42, wherein the rods extend axially along a length of the elastomeric layer and are circumferentially spaced apart from each other.

Example 44. The delivery apparatus of any example herein, particularly any of examples 40-43, wherein when the valve positioning structure is expanded to the radially expanded state upon inflation of the balloon, the rods move radially outwards relative to the shaft and the elastomeric layer expands radially, and when the balloon is deflated, the elastomeric layer radially contracts and urges the rods back to the radially collapsed state.

Example 45. The delivery apparatus of any example herein, particularly any one of examples 1-44, wherein the valve positioning structure comprises a first valve positioning structure, and the delivery apparatus further comprises a second valve positioning structure as recited in any of examples 1-44.

Example 46. The delivery apparatus of any example herein, particularly example 45, wherein the first valve positioning structure extends over a distal end portion of the balloon and the second valve positioning structure extends over a proximal end portion of the balloon.

Example 47. A delivery apparatus for delivering a prosthetic valve through vasculature of a patient, the delivery apparatus comprising: a handle; a shaft coupled to the handle and a balloon coupled to a distal end portion of the shaft, wherein the balloon is configured to be inflated from a first, deflated state to a second, radially expanded, inflated state, wherein the balloon has a valve mounting portion for mounting the prosthetic valve in a radially compressed state; and a valve positioning structure extending at least partially over an exterior surface of the balloon and having an inner member and an outer member, wherein the valve positioning structure is configured to radially expand from a radially collapsed state to a radially expanded state when the balloon is inflated and then radially collapse from the radially expanded state to the radially collapsed state when the balloon is deflated, wherein the outer member is configured to exert a radial compression force against the inner member to urge the inner member back to the radially collapsed state when the balloon is deflated.

Example 48. The delivery apparatus of any example herein, particularly example 47, wherein the valve positioning structure is positioned along a distal end portion of the balloon, and wherein the valve positioning structure is configured to limit distal movement of the prosthetic valve relative to the balloon.

Example 49. The delivery apparatus of any example herein, particularly either example 47 or example 48, further comprising a nose cone connected to the distal end portion of the shaft, wherein a distal portion of the valve positioning structure is connected to the nose cone.

Example 50. The delivery apparatus of any example herein, particularly example 49, wherein an inner surface of the distal portion of the valve positioning structure is bonded to an outer surface of the nose cone.

Example 51. The delivery apparatus of any example herein, particularly example 47, wherein the valve positioning structure is positioned along a proximal end portion of the balloon, and wherein the valve positioning is configured to limit proximal movement of the prosthetic valve relative to the balloon.

Example 52. The delivery apparatus of any example herein, particularly example 51, wherein the shaft comprises a first shaft, and the delivery apparatus further comprises a second shaft extending coaxially over the first shaft, and wherein a proximal portion of the valve positioning structure is connected to a distal end portion of the second shaft.

Example 53. The delivery apparatus of any example herein, particularly example 52, wherein the second shaft is movable axially relative to the first shaft and the balloon, and the valve positioning structure is configured to move the prosthetic valve relative to the balloon from an offset position to the valve mounting portion of the balloon.

Example 54. The delivery apparatus of any example herein, particularly any one of examples 47-53, wherein the inner member comprises a material that is more rigid than the outer member.

Example 55. The delivery apparatus any example herein, particularly of any of examples 47-54, wherein the inner member comprises a base and a plurality of prongs extending axially from the base, wherein the prongs extend at least partially over the exterior of the balloon and are configured to transition from the radially collapsed state to the radially expanded state when the balloon is inflated, and wherein the outer member is configured to urge the prongs back to the radially collapsed state when the balloon is deflated.

Example 56. The delivery apparatus of any example herein, particularly example 55, wherein each prong comprises a channel portion extending in the circumferential direction, and wherein the outer member is positioned in the channel portions of the prongs.

Example 57. The delivery apparatus of any example herein, particularly either example 55 or example 56, wherein each prong comprises a fixed end portion connected to the base and a free end portion opposite the fixed end portion.

Example 58. The delivery apparatus of any example herein, particularly any of examples 55-57, wherein the free end portion of each prong comprises a radially extending flange.

Example 59. The delivery apparatus of any example herein, particularly any one of examples 55-58, wherein the outer member comprises an elastomeric band or sleeve.

Example 60. The delivery apparatus of any example herein, particularly example 59, wherein the outer member is made of an elastomer.

Example 61. The delivery apparatus of any example herein, particularly any of examples 55-58, wherein the outer member comprises a coiled member.

Example 62. The delivery apparatus of any example herein, particularly example 61, wherein the outer member comprises coiled wire or band.

Example 63. The delivery apparatus of any example herein, particularly any of examples 61-62, wherein the outer member is made of a shape memory material and is shaped set in a radially collapsed state.

Example 64. The delivery apparatus of any example herein, particularly example 63, wherein the shape memory material comprises Nitinol.

Example 65. The delivery apparatus of any example herein, particularly any of examples 55-58, wherein the outer member comprises an annular band made of Nitinol.

Example 66. The delivery apparatus of any example herein, particularly example 65, wherein the annular band comprises a plurality of axial struts having opposing ends, each of which is connected to an adjacent end of an adjacent strut by a connecting portion of the band.

Example 67. The delivery apparatus of any example herein, particularly any of examples 65-66, wherein the outer member is shape set in a radially collapsed state.

Example 68. The delivery apparatus of any example herein, particularly any of examples 65-67, wherein the outer member is a closed annular band.

Example 69. The delivery apparatus of any example herein, particularly any of examples 55-68, wherein each prong has first and second interlocking features on opposite sides of the prong that engage adjacent interlocking features of adjacent prongs when the prongs are in the radially collapsed state.

Example 70. The delivery apparatus of any example herein, particularly example 69, wherein the first interlocking feature comprises a projection and the second interlocking feature comprises a notch shaped to receive a projection of adjacent prong.

Example 71. The delivery apparatus of any example herein, particularly any of examples 55-70, when dependent on example 52 or example 53, wherein the inner member is connected to the distal end portion of the second shaft.

Example 72. The delivery apparatus of any example herein, particularly any of examples 47-54, wherein the inner member comprises a plurality of axially extending rods and the outer member comprises an annular, elastomeric layer extending around the rods.

Example 73. The delivery apparatus of any example herein, particularly example 72, wherein the rods are made of a first material and the elastomeric layer is made of a second material, wherein the first material is stiffer than the second material.

Example 74. The delivery apparatus of any example herein, particularly any of examples 72-74, wherein the elastomeric layer and the rods comprise different materials that are coextruded together.

Example 75. The delivery apparatus of any example herein, particularly any of examples 72-74, wherein the rods extend axially along a length of the elastomeric layer and are circumferentially spaced apart from each other.

Example 76. The delivery apparatus of any example herein, particularly any of examples 72-74, when dependent on example 52 or example 53, wherein the distal end portion of the second shaft extends partially into a lumen of the valve positioning structure.

Example 77. The delivery apparatus of any example herein, particularly example 76, wherein the distal end portion of the second shaft is bonded to an inner surface of the valve positioning structure.

Example 78. The delivery apparatus of any example herein, particularly any of examples 47-77, in combination with the prosthetic valve mounted on the valve mounting portion in a radially compressed state.

Example 79. A delivery apparatus for delivering a prosthetic valve through vasculature of a patient, the delivery apparatus comprising: a handle; a shaft coupled to the handle and a balloon coupled to a distal end portion of the shaft, wherein the balloon is configured to be inflated from a first, deflated state to a second, radially expanded, inflated state, wherein the balloon has a valve mounting portion for mounting the prosthetic valve in a radially compressed state; and a valve positioning structure comprising a plurality of prongs extending at least partially over an exterior surface of the balloon and at least one biasing member, wherein the prongs are configured to radially expand from a radially collapsed state to a radially expanded state when the balloon is inflated and then radially collapse from the radially expanded state to the radially collapsed state under a biasing force of the biasing member when the balloon is deflated.

Example 80. The delivery apparatus of any example herein, particularly example 79, wherein each prong has a fixed end connected to a base of the valve positioning structure and a free end opposite the fixed end.

Example 81. The delivery apparatus of any example herein, particularly example 80, further comprising a second shaft extending coaxially over the first shaft, wherein the base is connected to a distal end portion of the second shaft.

Example 82. The delivery apparatus of any example herein, particularly example 79, further comprising a second shaft extending coaxially over the first shaft, wherein each prong extends from a distal end portion of the second shaft.

Example 83. The delivery apparatus of any example herein, particularly any of examples 79-82, wherein the at least one the biasing member comprises a plurality of biasing members, each of which is coupled to a respective prong.

Example 84. The delivery apparatus of any example herein, particularly example 83, wherein the biasing members comprise springs.

Example 85. The delivery apparatus of any example herein, particularly example 84, wherein the springs comprise tension springs.

Example 86. The delivery apparatus of any example herein, particularly any of examples 79-85, wherein a first end of each spring is coupled to one of the prongs, wherein a second end of the spring is coupled to an adjacent surface of the delivery apparatus.

Example 87. The delivery apparatus of any example herein, particularly example 86, wherein each spring is located radially between a respective prong and the adjacent surface of the delivery apparatus.

Example 88. The delivery apparatus of any example herein, particularly any of examples 86-87, when dependent on example 80 or example 81, wherein the springs are spaced axially from the fixed ends of the prongs.

Example 89. The delivery apparatus of any example herein, particularly any of examples 79-82, wherein the at least one biasing member is disposed around the prongs.

Example 90. The delivery apparatus of any example herein, particularly example 89, wherein the biasing member comprises an elastomeric band or sleeve.

Example 91. The delivery apparatus of any example herein, particularly example 90, wherein the biasing member is made of an elastomer.

Example 92. The delivery apparatus of any example herein, particularly example 89, wherein the biasing member comprises a coiled member.

Example 93. The delivery apparatus of any example herein, particularly example 92, wherein the biasing member comprises coiled wire or band.

Example 94. The delivery apparatus of any example herein, particularly any of examples 92-93, wherein the biasing member is made of a shape memory material and is shaped set in a radially collapsed state.

Example 95. The delivery apparatus of any example herein, particularly example 94, wherein the shape memory material comprises Nitinol.

Example 96. The delivery apparatus of any example herein, particularly example 89, wherein the biasing member comprises an annular band comprising a plurality of interconnected struts made of Nitinol.

Example 97. The delivery apparatus of any example herein, particularly example 96, wherein the struts comprise a plurality of axial struts having opposing ends, each of which is connected to an adjacent end of an adjacent strut by a connecting portion of the band.

Example 98. The delivery apparatus of any example herein, particularly any of examples 96-97, wherein the biasing member is shape set in a radially collapsed state.

Example 99. The delivery apparatus of any example herein, particularly any of examples 96-98, wherein the biasing member is a closed annular band.

Example 100. The delivery apparatus of any example herein, particularly any of examples 89-99, wherein the biasing member is disposed in a channel formed on outer surfaces of the prongs.

Example 101. The delivery apparatus of any example herein, particularly any of examples 79-100, in combination with the prosthetic valve mounted on the valve mounting portion in a radially compressed state.

Example 102. A delivery apparatus for delivering a prosthetic valve through vasculature of a patient, the delivery apparatus comprising: a handle; a first shaft coupled to the handle and extending therefrom; a second shaft coupled to the handle and extending therefrom, wherein the second shaft extends coaxially over the first shaft; a balloon mounted on a distal end portion of the first shaft, wherein the balloon is configured to be inflated from a first, deflated state to a second, radially expanded, inflated state, wherein the balloon has a valve mounting portion for mounting the prosthetic valve in a radially compressed state; and a distal tip portion extending from a distal end of the second shaft, wherein the distal tip portion is configured to be positioned over a proximal end portion of the balloon and expand radially from a radially collapsed state to a radially expanded state upon inflation of the balloon.

Example 103. The delivery apparatus of any example herein, particularly example 102, wherein the tip portion comprises a plurality of axially extending rods and an annular, elastomeric layer extending around the rods.

Example 104. The delivery apparatus of any example herein, particularly example 103, wherein the rods are made of a first material and the elastomeric layer is made of a second material, wherein the first material is stiffer than the second material.

Example 105. The delivery apparatus of any example herein, particularly any of examples 103-104, wherein the elastomeric layer and the rods comprise different materials that are coextruded together.

Example 106. The delivery apparatus of any example herein, particularly any of examples 103-105, wherein the rods extend axially along a length of the elastomeric layer and are circumferentially spaced apart from each other.

Example 107. The delivery apparatus of any example herein, particularly example 106, wherein the rods are parallel to each other.

Example 108. The delivery apparatus of any example herein, particularly any of examples 102-107, wherein when the balloon is inflated, the rods move radially outwards relative to the second shaft and the elastomeric layer expands radially, and when the balloon is deflated, the elastomeric layer radially contracts and urges the rods to a radially collapsed state.

Example 109. The delivery apparatus of any example herein, particularly any of examples 102-108, wherein when the prosthetic valve is mounted on the valve mounting portion in a radially compressed state, the tip portion is configured to abut a proximal end of the prosthetic valve to resist movement of the prosthetic valve relative to the balloon in a proximal direction.

Example 110. The delivery apparatus of any example herein, particularly any of examples 102-108, wherein the second shaft and the tip portion are axially moveable relative to the first shaft to push the prosthetic valve from a first position proximal to the valve mounting portion to a second position on the valve mounting portion.

Example 111. A delivery system for delivering a prosthetic valve through vasculature of a patient, the delivery system comprising: a radially expandable prosthetic valve; a delivery apparatus comprising: a handle; a shaft coupled to the handle and a balloon coupled to a distal end portion of the shaft, wherein the balloon is configured to be inflated from a first, deflated state to a second, radially expanded, inflated state, wherein the prosthetic valve is mounted on the balloon in a radially compressed state; an expandable distal shoulder disposed on an exterior surface of a distal end portion of the balloon; and an expandable proximal shoulder disposed on an exterior surface of a proximal end portion of the balloon; wherein the distal and proximal shoulders are configured to radially expand from a radially collapsed state to a radially expanded state when the balloon is inflated and then radially collapse from the radially expanded state to the radially collapsed state when the balloon is deflated.

Example 112. The delivery system of any example herein, particularly example 111, wherein the distal shoulder is positioned to limit distal movement of the prosthetic valve relative to the balloon.

Example 113. The delivery system of any example herein, particularly any of examples 111-112, wherein the proximal shoulder is positioned to limit proximal movement of the prosthetic valve relative to the balloon.

Example 114. The delivery system of any example herein, particularly any of examples 111-113, wherein the distal shoulder is coupled to a distal end portion of the shaft and forms an integral nose cone.

Example 115. The delivery system of any example herein, particularly any of examples 111-113, further comprising a nose cone coupled to a distal end portion of the shaft, wherein a distal portion of the distal shoulder is coupled to the nose cone.

Example 116. The delivery system of any example herein, particularly example 115, wherein the distal portion of the distal shoulder is bonded to an exterior surface of the nose cone.

Example 117. The delivery system of any example herein, particularly any of examples 111-116, wherein a maximum outer diameter of the distal shoulder is greater than an outer diameter of the radially compressed prosthetic valve.

Example 118. The delivery system of any example herein, particularly any of examples 111-117, wherein a maximum outer diameter of the proximal shoulder is greater than an outer diameter of the radially compressed prosthetic valve.

Example 119. The delivery system of any example herein, particularly any of examples 111-118, wherein the shaft comprises a first shaft and the delivery apparatus comprises a second shaft extending coaxially over the first shaft, wherein a proximal portion of the proximal shoulder is coupled to a distal end portion of the second shaft.

Example 120. The delivery system of any example herein, particularly any one of examples 111-119, wherein the proximal shoulder and/or the distal shoulder comprises an inner member and an outer member positioned radially outwards of the inner member.

Example 121. The delivery system of any example herein, particularly example 120, wherein the inner member comprises a plurality of prongs.

Example 122. The delivery system of any example herein, particularly any one of examples 120-121, wherein the inner member comprises a material that is more rigid than the outer member.

Example 123. The delivery system of any example herein, particularly any one of examples 111-119, wherein the proximal shoulder and/or the distal shoulder comprises an elastomeric sleeve.

Example 124. A delivery system for delivering a prosthetic valve through vasculature of a patient, the delivery system comprising: a radially expandable prosthetic valve; a delivery apparatus comprising: a handle; a shaft coupled to the handle and a balloon coupled to a distal end portion of the shaft, wherein the balloon is configured to be inflated from a first, deflated state to a second, radially expanded, inflated state, wherein the prosthetic valve is mounted on the balloon in a radially compressed state; an expandable shoulder disposed on an exterior surface of a portion of the balloon; wherein the shoulder is configured to radially expand from a radially collapsed state to a radially expanded state when the balloon is inflated and then radially collapse from the radially expanded state to the radially collapsed state when the balloon is deflated.

Example 125. The delivery system of any example herein, particularly example 124, wherein the shoulder is a distal shoulder disposed on a distal portion of the balloon and positioned to limit distal movement of the prosthetic valve relative to the balloon.

Example 126. The delivery system of any example herein, particularly any of examples 124-125, wherein the distal shoulder is coupled to a distal end portion of the shaft and forms an integral nose cone.

Example 127. The delivery system of any example herein, particularly any of examples 124-126, further comprising a nose cone coupled to a distal end portion of the shaft, wherein a distal portion of the distal shoulder is coupled to the nose cone.

Example 128. The delivery system of any example herein, particularly example 127, wherein the distal portion of the distal shoulder is bonded to an exterior surface of the nose cone.

Example 129. The delivery system of any example herein, particularly any of examples 124-128, wherein a maximum outer diameter of the distal shoulder is greater than an outer diameter of the radially compressed prosthetic valve.

Example 130. The delivery system of any example herein, particularly any of examples 124-129, wherein the shoulder is a proximal shoulder disposed on a proximal portion of the balloon and is positioned to limit proximal movement of the prosthetic valve relative to the balloon.

Example 131. The delivery system of any example herein, particularly example 130, wherein a maximum outer diameter of the proximal shoulder is greater than an outer diameter of the radially compressed prosthetic valve.

Example 132. The delivery system of any example herein, particularly any of examples 130-131, wherein the shaft comprises a first shaft and the delivery apparatus comprises a second shaft extending coaxially over the first shaft, wherein a proximal portion of the proximal shoulder is coupled to a distal end portion of the second shaft.

Example 133. The delivery system of any example herein, particularly any one of examples 124-132, wherein the shoulder comprises an inner member and an outer member positioned radially outwards of the inner member.

Example 134. The delivery system of any example herein, particularly example 133, wherein the inner member comprises a plurality of prongs.

Example 135. The delivery system of any example herein, particularly any one of examples 133-134, wherein the inner member comprises a material that is more rigid than the outer member.

Example 136. The delivery system of any example herein, particularly any one of examples 124-132, wherein the shoulder comprises an elastomeric sleeve.

Example 137. The delivery system of any example herein, particularly any one of examples 124-136, wherein the shoulder comprises a distal shoulder and a proximal shoulder, wherein the distal shoulder is disposed on a distal portion of the balloon and the proximal shoulder is disposed on a proximal portion of the balloon, and wherein the prosthetic valve is positioned between the distal and proximal shoulders.

Example 138. A delivery system for delivering a prosthetic valve through vasculature of a patient, the delivery system comprising: a radially expandable prosthetic valve; a delivery apparatus comprising: a handle; a shaft coupled to the handle and a balloon coupled to a distal end portion of the shaft, wherein the balloon is configured to be inflated from a first, deflated state to a second, radially expanded, inflated state, wherein the balloon has a valve mounting portion; and a valve positioning structure; wherein the prosthetic valve is mounted on the delivery apparatus is an radially compressed state offset from the valve mounting portion, wherein the valve positioning structure is configured to move the radially compressed prosthetic valve onto the valve mounting portion of the balloon such that the valve mounting portion extends at least partially over the balloon, and wherein the valve positioning structure is configured to radially expand from a radially collapsed state to a radially expanded state when the balloon is inflated and then radially collapse from the radially expanded state to the radially collapsed state when the balloon is deflated.

Example 139. The delivery apparatus of any example herein, particularly any one of examples 1-138, wherein the delivery apparatus is sterilized.

The features described herein with regard to any example can be combined with other features described in any one or more of the other examples, unless otherwise stated. For example, any one or more of the features of expandable valve positioning structure can be combined with any one or more features of another expandable valve positioning structure. As another example, any one or more features of one delivery apparatus can be combined with any one or more features of another delivery apparatus.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

We claim:

1. A delivery apparatus for delivering a prosthetic valve through vasculature of a patient, the delivery apparatus comprising:
a handle;
a shaft coupled to the handle and a balloon coupled to a distal end portion of the shaft, wherein the balloon is configured to be inflated from a first, deflated state to a second, radially expanded, inflated state, wherein the balloon has a valve mounting portion for mounting the prosthetic valve in a radially compressed state; and
a valve positioning structure extending at least partially over an exterior surface of the balloon and having a first end portion and a second end portion, wherein the valve positioning structure is configured to radially expand from a radially collapsed state to a radially expanded state when the balloon is inflated and then radially collapse from the radially expanded state to the radially collapsed state when the balloon is deflated.

2. The delivery apparatus of claim 1, wherein the valve positioning structure is positioned along a distal end portion of the balloon, and wherein the valve positioning structure is configured to limit distal movement of the prosthetic valve relative to the balloon.

3. The delivery apparatus of either claim 1, wherein the valve positioning structure comprises an integral nose cone that is connected to the distal end portion of the shaft distal to the balloon.

4. The delivery apparatus of claim 1, wherein the valve positioning structure is positioned along a proximal end portion of the balloon, and wherein the valve positioning structure is configured to limit proximal movement of the prosthetic valve relative to the balloon.

5. The delivery apparatus of claim 4, wherein the shaft comprises a first shaft, and the delivery apparatus further comprises a second shaft extending coaxially over the first shaft, and wherein the first end portion of the valve positioning structure is connected to a distal end portion of the second shaft.

6. The delivery apparatus of claim 5, wherein the second shaft is movable axially relative to the first shaft and the balloon, and the valve positioning structure is configured to move the prosthetic valve relative to the balloon from an offset position to the valve mounting portion of the balloon.

7. The delivery apparatus of claim 1, wherein a maximum outer diameter of the valve positioning structure is greater than an outer diameter of the prosthetic valve in the radially compressed state.

8. The delivery apparatus of claim 1, wherein the first end portion of the valve positioning structure has an outer diameter that is smaller than an outer diameter of the second end portion of the valve positioning structure.

9. The delivery apparatus of claim 1, wherein the valve positioning structure is elastically expandable and collapsible upon inflation and deflation of the balloon, respectively.

10. The delivery apparatus of claim 1, wherein the valve positioning structure stretches radially and circumferentially when the balloon is inflated and contracts radially and circumferentially when the balloon is deflated.

11. The delivery apparatus of claim 1, wherein the valve positioning structure is made of an elastomer.

12. The delivery apparatus of claim 1, wherein the valve positioning structure comprises an inner member and an outer member positioned radially outwards of the inner member.

13. The delivery apparatus of claim 12, wherein the inner member comprises a base and a plurality of prongs extending axially from the base, wherein the prongs extend at least partially over the exterior surface of the balloon and are configured to transition from the radially collapsed state to the radially expanded state when the balloon is inflated, and wherein the outer member is configured to urge the prongs back to the radially collapsed state when the balloon is deflated.

14. The delivery apparatus of claim 13, wherein the outer member comprises an elastomeric band or sleeve.

15. The delivery apparatus of claim 13, wherein the outer member comprises a coiled member.

16. A delivery apparatus for delivering a prosthetic valve through vasculature of a patient, the delivery apparatus comprising:
a handle;
a first shaft coupled to the handle and extending therefrom;
a second shaft coupled to the handle and extending therefrom, wherein the second shaft extends coaxially over the first shaft;
a balloon mounted on a distal end portion of the first shaft, wherein the balloon is configured to be inflated from a first, deflated state to a second, radially expanded, inflated state, wherein the balloon has a valve mounting portion for mounting the prosthetic valve in a radially compressed state; and
a distal tip portion extending from a distal end of the second shaft, wherein the distal tip portion is configured to be positioned over a proximal end portion of the balloon and expand radially from a radially collapsed state to a radially expanded state upon inflation of the balloon.

17. The delivery apparatus of claim 16, wherein when the prosthetic valve is mounted on the valve mounting portion in a radially compressed state, the tip portion is configured to abut a proximal end of the prosthetic valve to resist movement of the prosthetic valve relative to the balloon in a proximal direction.

18. The delivery apparatus of claim 16, wherein the second shaft and the tip portion are axially moveable relative to the first shaft to push the prosthetic valve from a first position proximal to the valve mounting portion to a second position on the valve mounting portion.

19. A delivery system for delivering a prosthetic valve through vasculature of a patient, the delivery system comprising:
a radially expandable prosthetic valve; and
a delivery apparatus comprising:
a handle;
a shaft coupled to the handle and a balloon coupled to a distal end portion of the shaft, wherein the balloon is configured to be inflated from a first, deflated state to a second, radially expanded, inflated state, wherein the prosthetic valve is mounted on the balloon in a radially compressed state; and
an expandable shoulder disposed on an exterior surface of a portion of the balloon;
wherein the shoulder is configured to radially expand from a radially collapsed state to a radially expanded state when the balloon is inflated and then radially collapse from the radially expanded state to the radially collapsed state when the balloon is deflated.

20. The delivery system of claim 19, wherein the shoulder is a distal shoulder disposed on a distal portion of the balloon and positioned to limit distal movement of the prosthetic valve relative to the balloon.

21. The delivery system of claim 19, wherein a maximum outer diameter of the distal shoulder is greater than an outer diameter of the radially compressed prosthetic valve.

22. The delivery system of claim 19, wherein the shoulder is a proximal shoulder disposed on a proximal portion of the balloon and is positioned to limit proximal movement of the prosthetic valve relative to the balloon.

23. The delivery system of claim 22, wherein a maximum outer diameter of the proximal shoulder is greater than an outer diameter of the radially compressed prosthetic valve.

24. The delivery system of claim 19, wherein the shoulder comprises a distal shoulder and a proximal shoulder, wherein the distal shoulder is disposed on a distal portion of the balloon and the proximal shoulder is disposed on a proximal portion of the balloon, and wherein the prosthetic valve is positioned between the distal and proximal shoulders.
